(19) United States
(12) Patent Application Publication
(10) Pub. No.: US 2026/0060681 A1
(43) Pub. Date: Mar. 5, 2026

(54) WOUND CLOSURE AND TISSUE COUPLING SYSTEMS AND METHODS

(71) Applicant: Vasorum Ltd., Dublin (IE)

(72) Inventors: James E. Coleman, Dublin (IE); John P. Healy, Co. Meath (IE)

(21) Appl. No.: 18/977,902

(22) Filed: Dec. 11, 2024

Related U.S. Application Data
(60) Provisional application No. 63/690,289, filed on Sep. 3, 2024.

(51) Int. Cl. A61B 17/11; A61B 17/00

(52) U.S. Cl. CPC A61B 17/11; A61B 2017/00477; A61B 2017/00606; A61B 2017/00646

(57) ABSTRACT

Surgical assemblies are provided, including an introducer sheath defining a central lumen and a blood signal port, an actuator having an elongate shaft extending distally therefrom, and a deployable coupler coupled to a distal end of the elongate shaft. The actuator is configured to couple to the introducer sheath at the proximal end such that the elongate shaft extends through the central lumen of the introducer sheath. The deployable coupler has a plurality of proximal slits configured to form a proximal wing and a plurality of distal slits configured to form a distal wing, and is disposed distally of the introducer sheath when the actuator is coupled to the introducer sheath. A blood signal flow inlet is defined between the introducer sheath and the deployable coupler such that blood can enter the blood signal flow inlet, travel the central lumen, and exit via the blood signal port.

### CROSS-REFERENCE TO RELATED APPLICATIONS

This application claims the benefit of U.S. Provisional Application No. 63/690,289, filed Sep. 3, 2024, which is incorporated herein by specific reference.

### FIELD

Methods and device for wound closure and creating an anastomosis are provided.

### BACKGROUND

Many surgical procedures involve creating punctures in tissue at a surgical site or anastomosing tissue to create an anastomosis, such as a bowel anastomosis or an arteriovenous fistula between an artery and a vein. In the case of fistula formation, the purpose of such a connection is to create either a high flow connection or to create a pathway for blood around an obstruction in a replacement conduit or bypass. The conduit or bypass can generally be a vein, artery, or prosthetic graft.

An anastomosis can be created during a surgical procedure by bringing two vessels or conduits—such as bowel 200—into direct contact with each other and then joining them with sutures, clips, or other means. The anastomosis can be end-to-end, end-to-side, or side-to-side. When done with blood vessels, the anastomosis is typically elliptical in shape and is joined by hand with a suture. Other methods of anastomosis creation can involve carbon dioxide lasers, prostheses, clips, and stents. One type of fistula, an arteriovenous fistula, is created by connecting an artery to a vein. This type of connection can be used for hemodialysis, an increase in exercise tolerance, the treatment of hypertension, maintenance of an opening in an artery or vein, as an access path for chemotherapy, and others.

Various apparatuses have been proposed for percutaneously sealing tissue openings or for joining hollow structures in a patient's body, including biodegradable plugs, sutures, surgical fasteners, and other devices. However, these devices and associated methods have a multitude of shortcomings, including surgical risks, high failure rates, complexity, and more. Accordingly, there remains a need for improved devices and associated methods for closing tissue punctures and anastomosing tissue structures in a patient's body, or for deploying such devices in a simple and effective manner.

### SUMMARY

In general, methods and systems for creating an anastomosis and wound closure are provided.

In an embodiment, a surgical assembly is provided. The surgical assembly can include an introducer sheath defining a central lumen, an actuator having an elongate shaft extending distally therefrom, and a deployable coupler coupled to a distal end of the elongate shaft. The introducer sheath can include a blood signal port located at a proximal end thereof. The actuator can be configured to couple to the introducer sheath at the proximal end such that the elongate shaft extends through the central lumen of the introducer sheath. The deployable coupler can have a plurality of distal slits configured to form a distal wing and a plurality of proximal slits configured to form a proximal wing and can be distally of the introducer sheath when the actuator is coupled to the introducer sheath. A blood signal flow inlet can be defined between the introducer sheath and the deployable coupler when introducer sheath and the actuator are coupled together such that blood can enter the blood signal flow inlet disposed within tissue, travel the central lumen, and exit via the blood signal port disposed externally of the tissue.

The surgical assembly can vary in a number of ways. For example, the deployable coupler can define a central coupler lumen in fluid communication with the central lumen. In some variations, the introducer sheath, the actuator, and the deployable coupler can be deliverable into a body lumen via a guidewire configured to extend through the central lumen of the introducer sheath, the elongate shaft of the actuator, and the central coupler lumen of the deployable coupler. In other variations, the surgical assembly can include a plug tool configured to deploy a plug within the central coupler lumen of the deployable coupler to prevent fluid flow therethrough. For example, the deployable coupler can include a press ring centrally disposed between the proximal slits and the distal slits. For example, the actuator can be configured to deploy the proximal wing via a torsion and compressive forces applied in a first rotational direction to the deployable coupler. In some variations, the actuator can be configured to deploy the distal wing via a torsion force applied in a second rotational direction to the deployable coupler opposite the first direction. For example, the blood signal inlet can be disposed proximally of the proximal slits. For example, the proximal wing and the distal wing can be reversibly deployable.

In an embodiment, a surgical assembly is provided. The surgical assembly can include an actuator having an elongate shaft and an outer shaft disposed around the elongate shaft, and a deployable coupler coupled to a distal end of the elongate shaft and at a position distal of the slide plate. The outer shaft can be slidable relative to the elongate shaft, the outer shaft abutting a slide plate disposed at a distal end of the outer shaft. The outer shaft can be configured to slide distally to cause the slide plate to join with the deployable coupler and capture tissue therebetween. The actuator can be configured to eject the joined deployable coupler and the slide plate as a unit.

The surgical assembly can vary in a number of ways. For example, the deployable coupler can include a plurality of distal slits formed therein configured to form a distal wing. For example, the outer shaft can include a proximal region and a distal region, and a diameter of the distal region can be greater than a diameter of the proximal region. In some variations, the diameter of the distal region is substantially equal to a diameter of the slide plate. In other variations, the distal region flares outward from the proximal region. For example, the deployable coupler can define a central coupler lumen through which fluid is configured to flow. For example, the elongate shaft can define a central lumen therethrough. In some variations, the deployable coupler can define a central coupler lumen, and the central lumen and the central coupler lumen can be configured to receive a penetrator therethrough. The penetrator can be configured to extend beyond a distal end of the deployable coupler and to penetrate tissue and create an opening therein such that the deployable coupler can be advanced through the opening. For example, the actuator can be configured to deploy the distal wing via a torsion and compressive forces applied in a first rotational direction to the deployable coupler. In some variations, the actuator is configured to deploy the slide plate via a torsion force applied in a second rotational direction to the deployable coupler opposite the first direction a and/or a longitudinal force applied to the deployable coupler.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

These and other features will be more readily understood from the following detailed description taken in conjunction with the accompanying drawings, in which:

FIG. 1 is a perspective view of an actuator according to a first variation;

FIG. 2A is a perspective view of an actuator according to a second variation;

FIG. 2B is a perspective view of components of the actuator of FIG. 2A;

FIG. 2C is a partial cross-sectional view of an outer shaft hub of the actuator of FIG. 2A;

FIG. 2D is a partial side view of a coupler usable with the actuator of FIG. 2A;

FIG. 2E is a perspective view of the coupler of FIG. 2D is a deployed configuration without Slide Plate;

FIG. 2F is an exploded view of the coupler of FIG. 2E;

FIG. 2G is a perspective view of a slide plate usable with the actuator of FIG. 2A;

FIG. 2H is a rear perspective view of the slide plate of FIG. 2G;

FIG. 2I is an exploded view of the slide plate of FIG. 2G;

FIG. 2J is a partial perspective view of the components of FIG. 2A coupled together;

FIG. 3 is a perspective view of a plug tool 70 according to an embodiment;

FIG. 4 is a representation of a medical procedure utilizing the actuator of FIG. 1 depicting insertion of the actuator into an introducer sheath;

FIG. 5 is a representation of the medical procedure of FIG. 4 depicting the coupled actuator and introducer sheath;

FIG. 6 is a representation of the medical procedure of FIG. 4 depicting imaging of the coupler of FIG. 1;

FIG. 7 is a representation of the medical procedure of FIG. 4 depicting a blood signal;

FIG. 8 is a representation of the medical procedure of FIG. 4 depicting partial withdrawal of the actuator;

FIG. 9 is a representation of the medical procedure of FIG. 4 depicting elevation of the actuator and blood signal;

FIG. 10 is a representation of the medical procedure of FIG. 4 depicting deployment of a distal wing of the coupler and blood signal;

FIG. 11 is a representation of the medical procedure of FIG. 4 depicting tensioning of the actuator and deployment of a proximal wing of the coupler;

FIG. 12 is a representation of the medical procedure of FIG. 4 depicting injection of contrast media;

FIG. 13 is a representation of the medical procedure of FIG. 4 depicting a tray supporting the actuator of FIG. 1 during an exemplary procedure;

FIG. 14 is a representation of the medical procedure of FIG. 4 depicting removal of a contrast port of the actuator of FIG. 1;

FIG. 15 is a representation of the medical procedure of FIG. 4 depicting a rear perspective view of a central lumen of the actuator of FIG. 1;

FIG. 16 is a representation of the medical procedure of FIG. 4 depicting coupling of the plug tool of FIG. 3 with the actuator of FIG. 1;

FIG. 17 is a representation of the medical procedure of FIG. 4 depicting deployment of a plug via the plug tool of FIG. 16;

FIG. 18 is a representation of the medical procedure of FIG. 4 depicting decoupling of the plug tool of FIG. 16;

FIG. 19 is a representation of the medical procedure of FIG. 4 depicting deployment of the coupler of FIG. 11;

FIG. 20 is a representation of an improper deployment of a coupler via the actuator of FIG. 1;

FIG. 21 is a representation of a removal process step of the coupler of FIG. 20 depicting removal of a contrast port assembly and a guidewire from a body lumen;

FIG. 22 is a representation of the removal process of FIG. 21 depicting the insertion of a stiff guidewire;

FIG. 23A is a representation of the removal process of a cap during a coupler collapse process;

FIG. 23B is a representation of an exposed spring release mechanism;

FIG. 23C is a representation of actuation of the spring release mechanism of FIG. 23B;

FIG. 24 is a representation of the removal process of FIG. 21 depicting collapsing of the coupler of FIG. 20;

FIG. 25 is a representation of the removal process of FIG. 21 depicting the retraction of the flattened coupler of FIG. 20 from the puncture site;

FIG. 26 is a representation of the removal process of FIG. 21 depicting removal of the actuator, coupler, and sheath;

FIG. 27 is a representation of a deployment process of a coupler during a medical procedure using the actuator of FIG. 2A;

FIG. 28 is a representation of the medical procedure of FIG. 27 depicting partial retraction of the actuator until cessation of the blood signal;

FIG. 29 is a representation of the medical procedure of FIG. 27 depicting elevation of the actuator;

FIG. 30 is a representation of the medical procedure of FIG. 38 depicting deployment of a distal wing of the coupler;

FIG. 31 is a representation of the medical procedure of FIG. 27 depicting retraction of the actuator against a puncture site;

FIG. 32 is a representation of the medical procedure of FIG. 27 depicting actuation of the actuator to deploy the slide plate;

FIG. 33 is a representation of the medical procedure of FIG. 27 depicting injection of contrast media;

FIG. 34 is a representation of the medical procedure of FIG. 27 depicting deployment of the coupler and removal of the actuator;

FIG. 35 is a representation of a coupler removal process depicting collapsing of the distal wing of the coupler;

FIG. 36 is a representation of the coupler removal process of FIG. 35 depicting removal of the actuator and coupler;

FIG. 37 is an array of instruments including the actuator of FIG. 2A used to deliver an implant in a patient during a medical procedure;

FIG. 38 is a representation of a positioning step of an anastomosis medical process using the actuator of FIG. 2A;

FIG. 39 is a representation of a penetration step of the medical process of FIG. 38;

FIG. 40 is a representation of a penetration step of the medical process of FIG. 38;

FIG. 41 is a representation of a distension and advancement step of the medical process of FIG. 38;

FIG. 42 is a representation of the advancement of the coupler of the medical process of FIG. 38;

FIG. 43 is a representation of a deployment step of the medical process of FIG. 38;

FIG. 44 is a representation of a retraction step of the medical process of FIG. 38;

FIG. 45 is a representation of a coupling step of the medical process of FIG. 38;

FIG. 46 is a representation of an examination step of the medical process of FIG. 38;

FIG. 47 is a representation of a deployment step of the medical process of FIG. 38;

FIG. 48 is a representation of a positioning step of the medical process of FIG. 38;

FIG. 49 is a representation of a penetration step of the medical process of FIG. 38;

FIG. 50 is a representation of a deployment step of the medical process of FIG. 38;

FIG. 51 is a representation of an actuation step of the medical process of FIG. 38;

FIG. 52 is a representation of a deployment step of the medical process of FIG. 38; and

FIG. 53 is a representation of an inspection step of the medical process of FIG. 38.

It is noted that the drawings are not necessarily to scale. The drawings are intended to depict only typical aspects of the subject matter disclosed herein, and therefore should not be considered as limiting the scope of the disclosure.

### DETAILED DESCRIPTION

Certain illustrative embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting illustrative embodiments and that the scope of the present invention is defined solely by the claims. The features illustrated or described in connection with one illustrative embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape.

Surgical assemblies for use with anastomotic couplers and closure couplers are provided. In general, the surgical assembly can include an actuator device configured to deploy an anastomotic coupler within a patient to join and fluidly link tissue. The actuator device can include a handle having an elongate shaft extending distally therefrom. A distal end of the elongate shaft can have an anastomotic coupler affixed thereto, and the handle can be actuated to cause the affixed coupler to reversibly deploy one or more proximal and/or distal wing to couple the tissue therebetween. The coupler can then be decoupled from the elongate shaft. In the case of the closure coupler, prior to deployment of the one or more proximal and/or distal wing(s), the surgical assembly can employ a blood signal, which can be used to determine a position and/or orientation of the coupler relative to tissue in order to ensure proper deployment of the coupler. Depending upon the position of the coupler, blood can flow through between the coupler and elongate shaft and up the elongate shaft to provide a surgeon with a visual indicator of the position of the coupler.

In certain embodiments, the coupler can have a large, centrally disposed bore. The bore can facilitate fluid flow between joined regions of tissue, as may be needed for various surgical procedures. When fluid flow through the coupler is not desired, a plug can be advanced through the actuator device and into the large bore. The plug can then be permanently or reversibly affixed to the large bore to prevent fluid flow therethrough.

The closure assembly can be used in various surgical procedures. For example, the closure assembly can be used for the percutaneous closure of the common femoral arteriotomy or venotomy following diagnostic and/or interventional therapeutic intra-arterial procedures, such as peripheral or coronary angiography, arterial stents, balloon angioplasty, and atherectomy procedures where the arteriotomy is in the common femoral artery and closure assemblies have been used. Further, the closure assembly can be used in additional procedures, including, for example, in procedures promoting weight loss and/or the treatment of Type-2 diabetes.

Exemplary large bore closure assemblies are shown and described. These large bore closure assemblies can generally include a guidewire, an introducer sheath, an actuator, a deployable coupler, and a plug tool, and additional components or modified components may also be included in large bore closure assemblies. Reference is now made to large bore closure assemblies generally comprising these listed components, and two variations of actuator assemblies.

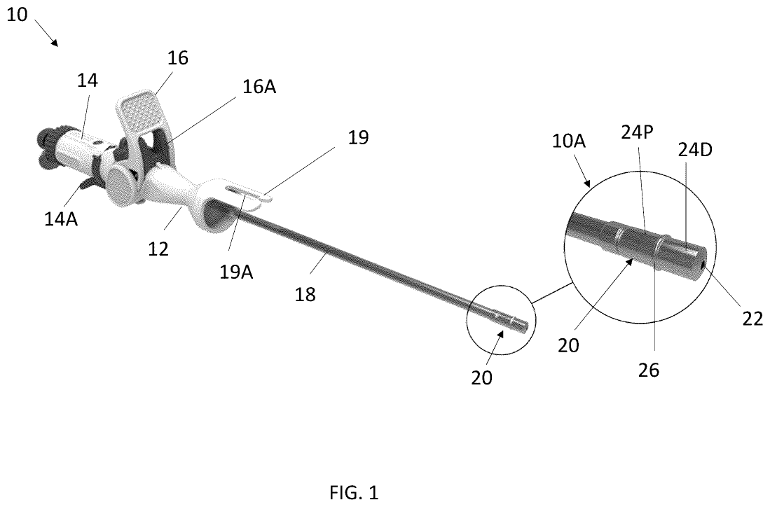

FIG. 1 depicts an actuator 10, according to a first variation, in greater detail. The actuator can generally include a body 12, a handle 14, an ejection lever 16, and a guide tube 18 extending distally from the body 12. A deployable coupler 20, shown in the form of a large bore coupler 20, can be removably affixed to a distal end of the guide tube 18. The components of the actuator 10 can vary in form as required, but as shown, the body 12 occupies a substantially cylindrical space. The handle 14 can extend proximally from the body 12 and be rotatably coupled thereto. Actuation of the handle 14 can transform the deployable coupler 20 as will be described below. The ejection lever 16 extends from the body 12 substantially perpendicular thereto. The ejection lever 16 can be pivoted from its upright position, depicted in FIG. 1, to position in which it is more aligned with the body 12 and the handle 14. Upon pivot, the ejection lever 16 can detach the deployable coupler 20 from its engagement with the guide tube 18, such as during a procedure. The actuator 10 can also include a removable locking tab 14A configured to prevent premature actuation of the handle 14, and a removable lever lock 16A configured to prevent premature actuation of the ejection lever 16.

As introduced above, the guide tube 18 can be substantially tubular and can couple a large bore coupler 20 on an end thereof. The inset 10A of FIG. 1 illustrate a close-up view of the end of the guide tube 18 having a large bore coupler 20 affixed thereto. The coupler 20 can have a generally cylindrical shape and define a central lumen 22 therethrough about a longitudinal axis thereof. The coupler 20 can be divided into a proximal region having a plurality of proximal slits 24P and a distal region having a plurality of distal slits 24D. A press ring 26 can be centrally disposed between the proximal and distal regions and the proximal and distal slits 24P, 24D. Upon actuation of the handle 14, the proximal and distal regions can be deployed and transformed into splayed wings, folding about the proximal and distal slits 24P, 24D respectively. This deployment will be described in greater detail below, but each wing can be reversibly deployed in parallel or in sequence, depending upon the variation.

As introduced above, the actuator 10 can include a sheath retainer with prongs 19 extending from the body 12 and proximate to the guide tube 18 configured to couple to a sheath, described in more detail below. In some embodiments, the sheath retainer can include a central track 19A having a plurality of engagement zones (not shown) configured to engage a sheath at a plurality of distances, thereby allowing for the guide tube 18 to be inserted into a sheath at substantially discrete insertion depths to facilitate blood flow through a blood signal outlet, described below. The actuator 10 can have a variable number of engagement zones, such as one, two, three, or more. The assembly can also include a removable sheath stop configured to prevent over-insertion of the guide tube 18 into a sheath. The removable sheath stop can be coupled to the actuator near the sheath retainer, and it can block the more-proximal engagement zone(s) to prevent over-insertion of the guide tube 18 into a sheath. More than one removable sheath stop can be used if more than one more-proximal engagement zone is used. When additional depth is required, the removable sheath stop can be decoupled from the actuator 10 to expose the more-proximal engagement zone(s). After coupling with a sheath, the guide tube 18 can then be inserted further into a sheath.

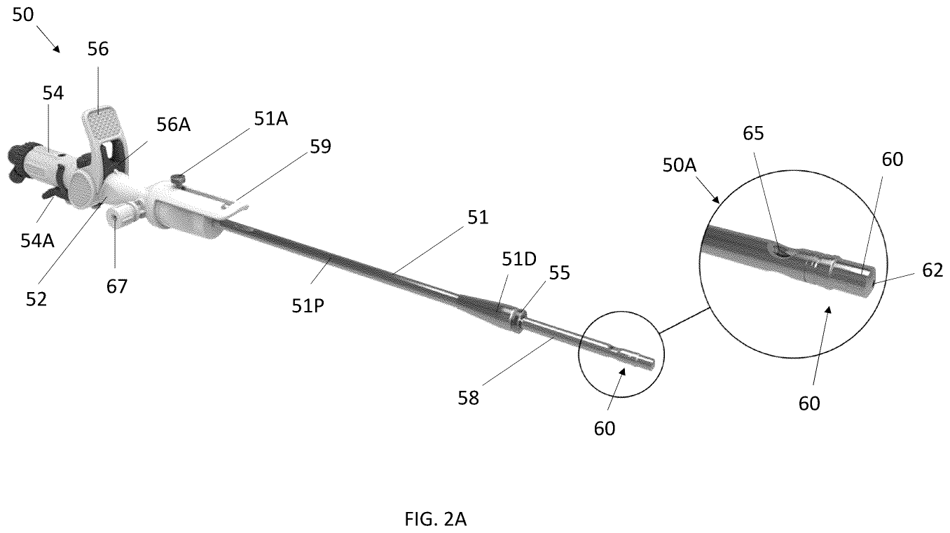

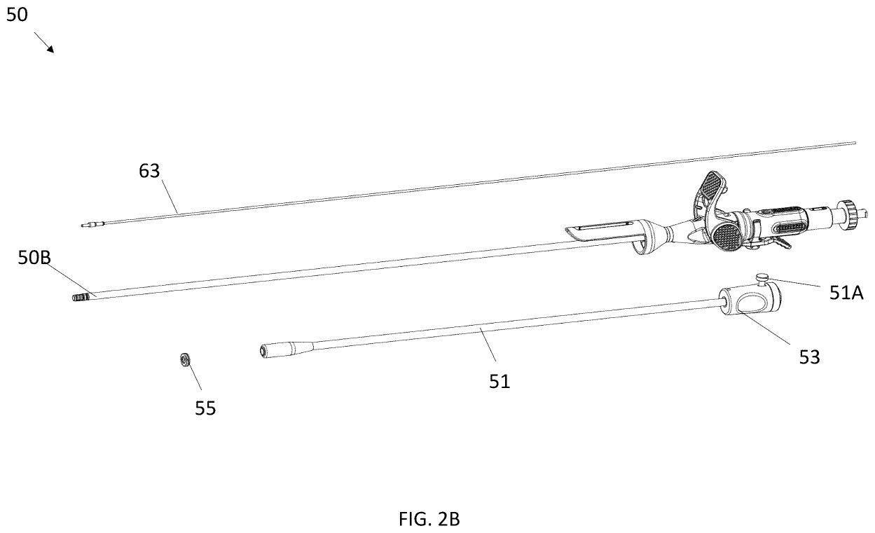

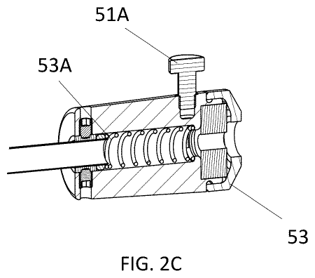

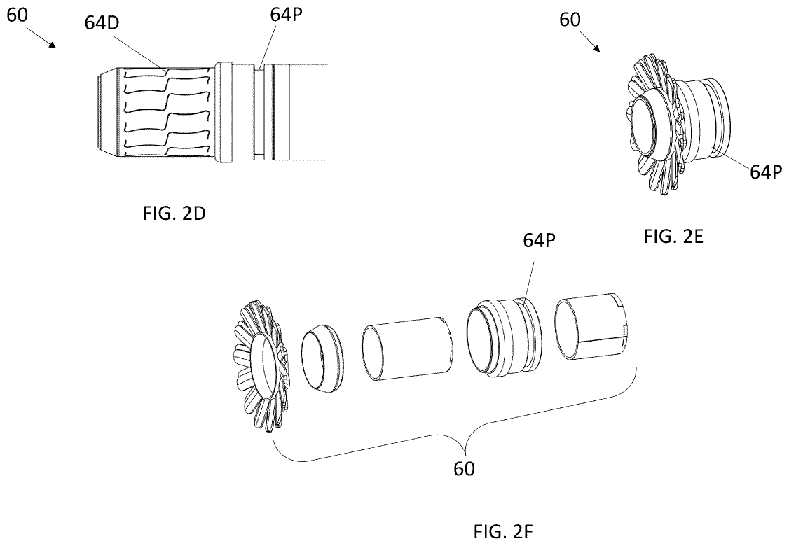

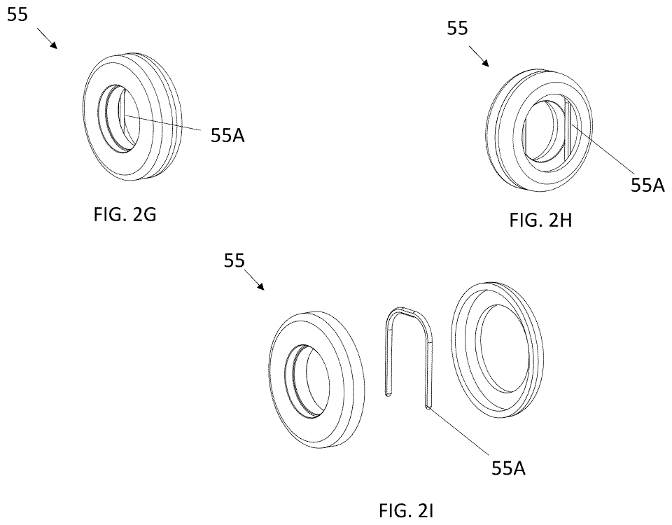

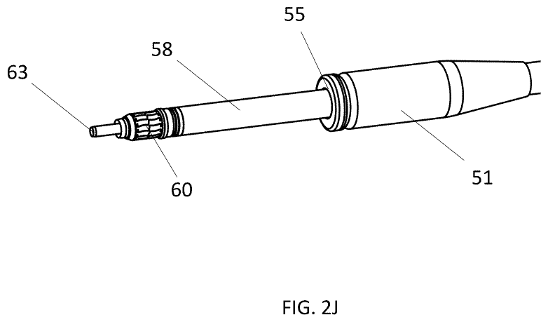

FIG. 2A depicts an actuator 50, according to a second variation, in greater detail. This second variation is similar to the first variation of FIG. 1 in many ways, and for brevity, like components will not be described again. Generally, the actuator 50 includes a body 52, handle 54 and locking tab 54A, and ejection lever 56 and lever lock 56A. A guide tube 58 extends distally from the body 52 and includes a coupler 60 removably affixed thereto. The second variation differs in a few ways. For example, the actuator 50 includes an outer sheath 51 including a proximal region 51P of substantially similar diameter and a flared distal region 51D having a diameter greater than a diameter of the proximal region 51P. The outer sheath 51 is disposed around the guide tube 58 of the actuator 50. Immediately distally of the flared distal region 51D is a slide plate 55. The actuator 50, instead of prongs, includes a track 59, which can receive a corresponding guide 51A of the outer sheath 51. In operation, the guide 51A can be moved within the track 59 as the outer sheath 51 is slide distally relative to the guide tube 58. The coupler 60, with central lumen 62 also varies in that it does not include proximal slits to form a proximal wing, only distal slits to form a distal wing. A blood signal inlet 65 can be located proximally of the coupler 60 as part of the guide tube 58, and a blood signal outlet 67 can be disposed on a side of the body 52 in fluid communication with the blood signal inlet 65. The coupler 60 is depicted in greater detail in the inset 50A.

In operation, the guide 51A can be moved distally through the track 59 to slide the outer sheath 51 and the slide plate 55 distally. This action can cause the slide plate 55 to couple to the coupler 60. Instead of a proximal wing, the coupler 60 can include the slide plate 55, which can be used to couple the coupler 60 to tissue as desired for a surgical procedure. Additional details of the actuator 50 and coupler 60 are described below. Further, an exemplary procedure involving the actuator 50 and coupler 60 is also described below.

FIG. 2B depicts the actuator 50 and several of its components separated out from each other. From top to bottom, the figure depicts a penetrator shaft 63, a base unit 50B of the actuator 50 including coupler 60, the outer sheath 51, and the slide plate 55. The penetrator shaft 63 can be slid down a central lumen of the actuator 50 during a procedure to penetrate tissue, as will be described in greater detail below. The outer sheath 51, seen in isolation, includes its guide 51A extending from a proximal receiver 53, which can be received by the base unit 60A as well, which, as explained above, can be received in the track 59 of the base unit 60A. The slide plate 55 is described in additional detail with reference to FIGS. 2D-F.

FIG. 2C depicts a cross-sectional view of the proximal receiver 53. In this view, a spring 53A is shown, which can 1) provide a user with feedback in the form of a counter force and 2) provide the required force across variations in guide tube length when the outer sheath 51 is slid distally to couple the slide plate 55 with the coupler 60 during a procedure.

As described previously with respect to the other variations, the coupler 60 can be deployed within a patient's body. The coupler 60 is depicted in FIGS. 2D-2F various stages, including pre-deployment, deployment, and an exploded view. The pre-deployment stage is depicted in FIG. 2D. Distal slits 64D are defined in an exterior of the coupler 60. A proximal groove 64P is defined in a rear of the coupler 60, and this proximal groove 64P can receive a corresponding clip in the slide plate 55, described below. In the deployed stage, of FIG. 2E, the distal wing has been deployed to splay outward from the center of the coupler. The coupler 60 can be actuated, such as via a torsion force applied at the handle 54 in conjunction with a simultaneous longitudinal force to fold the coupler inward upon itself. The distal slits 64D define boundaries of each petal in the wing. The exploded view of FIG. 2F depicts the components of the coupler 60. From left to right, these components include the distal wing, a core pin, a portion of the ejection tube, a slide pin, and a portion of the fused internal crown. The proximal groove 64P is still shown in this view.

FIGS. 2G-2I depicts several views of the slide plate 55, including two complete views and an exploded view below. The complete views of FIGS. 2G and 2H depict front perspective and rear perspective views respectively. A spring clip 55A, which is received within the proximal groove 64P of the coupler 60 can be seen in each of these figures. In the exploded view, the components from left to right include a slide plate housing, the spring clip, and a spring clip retainer. When the slide plate 55 is slid distally about the guide tube 58, the spring clip 55A, splayed slightly outward, will eventually snap into place in the proximal groove 64P to couple the slide plate 55 with the coupler 60.

When combined, components of the actuator 50 including the guide tube 58 with coupler 60, the outer sheath 51, the slide plate 55, and the penetrator shaft 63 can nest as shown in FIG. 2J (pre-deployed phase).

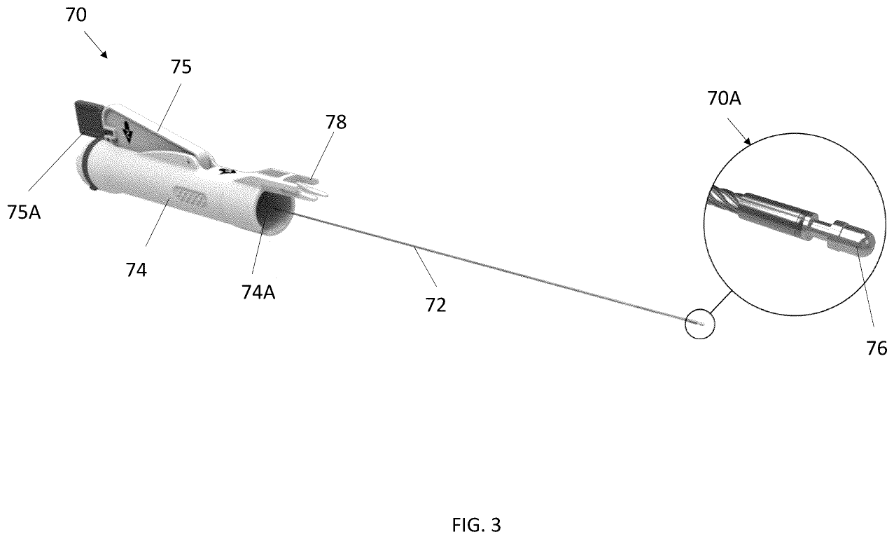

FIG. 3 depicts the plug tool 70 in greater detail. The plug tool 70 can be used to plug the central lumen of a coupler (e.g., coupler 20, 60) in order to prevent or occlude fluid flow therethrough. While the plug tool 70 may not be needed in surgical procedures where occlusion or prevention of fluid flow is not desired, the plug tool 70 can provide additional versatility for the treatment of various ailments. The illustrated plug tool 70 includes a substantially cylindrical plug tool 70 handle having a plug shaft 72 extending distally therefrom. The plug tool 70 handle 74 can include a distal crevice 74A from which the plug shaft 72 extends, and the distal crevice 74A can be sized to removably receive the handle 14, 54 of the actuator 10, 50. An ejectable plug 76, seen especially in inset 70A, can be removably affixed to a distal end of the plug shaft 72. The handle 74 can also include a lever 75 extending from a side thereof. Upon actuation, the lever 75 can be configured to eject the ejectable plug 76 from the distal end of the plug shaft 72. To prevent premature ejection and also to retain the lever 75 in its pre-deployment position, a plug lever lock 75A can be coupled to a proximal end of the handle 74 and can interfere with actuation of the lever 75 until an intended time during a surgical procedure. A set of prongs 78 can extend from the distal end of the handle 74 outside of the distal crevice 74A. The prongs 78 can be shaped and configured to couple with the removable lever lock 16A, 56A on the actuator 10, 50 when the plug tool 70 is affixed to the actuator 10, 50, thereby securing the plug tool 70 to the actuator 10, 50.

During a surgical procedure, as introduced above, the plug tool 70 can be coupled to the actuator 10, 50 and used to plug the central lumen 22, 62 of the coupler 20, 60 in order to prevent or occlude fluid flow therethrough. After an optional guidewire has been removed from the actuator 10, 50, the plug tool 70 can be extended, plug first, into the central lumen 22, 62 of the coupler 20, 60. The plug tool 70 can be inserted until the handle 14, 54 of the actuator 10, 50 is secured within the crevice of the plug tool 70 and until the prongs 78 couple with the removable lever lock 16A, 56A. At this depth, the ejectable plug 76 can be disposed centrally within the central lumen 22, 62 of the coupler 20, 60. During a removal process, the plug tool 70 can be decoupled from the actuator 10, 50. Decoupling the plug tool 70 from the actuator 10, 50 can leave the prongs 78 coupled to the removable lever lock 16A, 56A such that removal of the plug tool 70 also removes the removable lever lock 16A, 56A in one stroke.

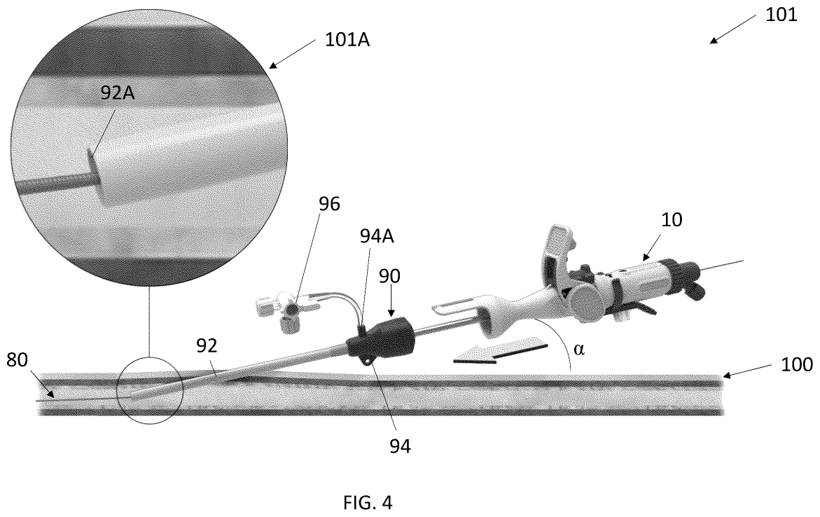

FIGS. 4-19 depict an example procedure using a guidewire 80, a sheath 90, the actuator 10, the deployable coupler 20, and the plug tool 70 acting within a body lumen 100 100 of a patient. In general, the procedure depicts the deployment of the deployable coupler 20 by the actuator 10 within body lumen 100.

A typical guidewire 80 can be disposed within the body lumen 100 via a procedural sheath. The guidewire 80 can be any standard-type guidewire known to those in the art. The guidewire 80 can be pre-inserted into a tissue and/or a cavity to aid in guiding surgical tools to a surgical site or site of interest. While the guidewire 80 can vary in specific form, in some embodiments, the guidewire 80 can have a diameter between approximately 0.01 and 0.05 inches. For example, the guidewire 80 can have a diameter of approximately 0.035 inches.

An exemplary introducer sheath is depicted with the surgical procedure of FIGS. 4-19. The sheath 90, as introduced above, can include an elongate shaft 92 attached to a hub 94 at a proximal end thereof. The elongate shaft 92 can define an inner lumen 92A. The hub 94 can be flared in shape and can have a port 94A extending from one side that leads to a valve assembly 96 for use during a surgical procedure as a blood signal. The port 94A and valve assembly 96 can be configured to provide a connection point for coupling the sheath 90 with an actuator.

When coupled thereto, an elongate shaft of an actuator can extend from the proximal end of the sheath 90 and through the inner lumen 92A. A coupler (e.g., coupler 20, 60) can extend distally beyond the sheath 90 when the actuator is coupled therewith. A dilator (not shown) can be placed within the sheath 90 to ensure the sheath 90 retains a desired configuration during insertion into a patient. The sheath 90 can come in various sizes, and each size sheath can be suited to close a range of puncture sizes.

The guidewire 80 protrudes from the distal end of the sheath 90 and into the body lumen 100. The initial placement of the sheath 90 and body lumen 100 can take place using various known techniques, which are not detailed herein. Once the guidewire is in proper position, the procedural sheath can be removed from the body lumen 100.

The sheath 90 is inserted over the guidewire 80 and into the body lumen 100 with the guidewire 80 protruding therefrom. Due to the sheath 90 being elongate, to ensure the sheath 90 does not bottom-out in the body lumen 100, it can be introduced at an entry angle relative to the body lumen 100. This entry angle can vary. For example, the entry angle can be less than about 35 degrees relative to the body lumen 100.

When the sheath 90 is in place, both the dilator and the guidewire 80 can be removed from the body lumen. Once both the guidewire 80 and dilator are fully withdrawn, a stepped guidewire straightener can be inserted into the port of the sheath and with a stiff variation of the guidewire 80 inserted therein. The stepped straightener and guidewire 80 can operate similarly to a needle and thread, where the stepped straightener maintains a straight orientation of the guidewire 80 so that it can be fed through the stepped straightener and sheath 90 and into the body lumen 100. Once the guidewire 80 is in position, the stepped guidewire straightener can be removed. The guidewire 80 remains in place.

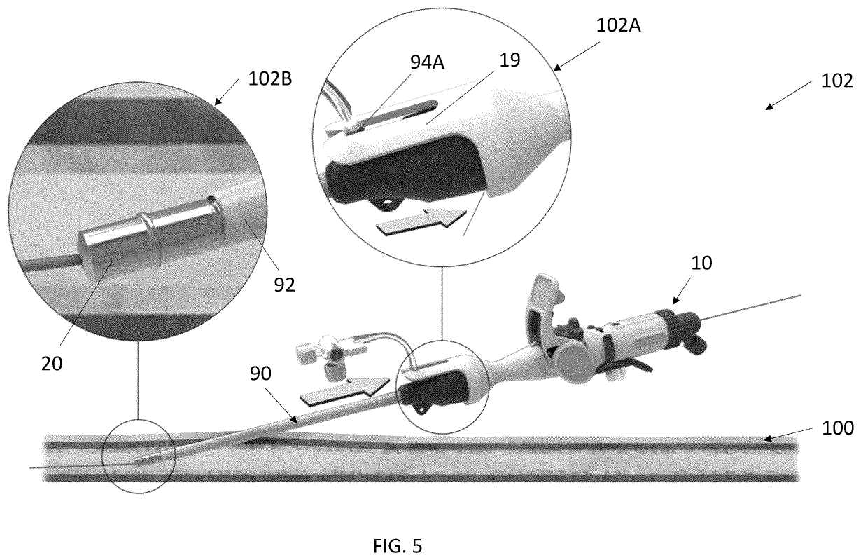

FIG. 4 depicts an insertion step 101 with the actuator 10 being inserted through the sheath 90 along the guidewire 80 and into the body lumen 100. Similar to the insertion of the sheath, the actuator 10 can be inserted at an angle α relative to the body lumen 100 so that the actuator 10 does not bottom-out in the body lumen 100 and potentially cause damage to the tissue. The angle of insertion α of the actuator 10 can vary. For example, the angle of insertion α can be less than about 45 degrees relative to the body lumen 100. The inset 101A of FIG. 4 depicts the guidewire 80 protruding from the sheath 90. Eventually, the prongs 19 of the actuator 10 will contact the port 94A of the sheath 90 and connect as shown in FIG. 5 in a coupling step 102. The inset 102A depicts this interaction in greater detail. The prongs 19 couple to the port 94A and the coupler 20 fully extends beyond the end of the sheath 90 as shown in the inset 102B.

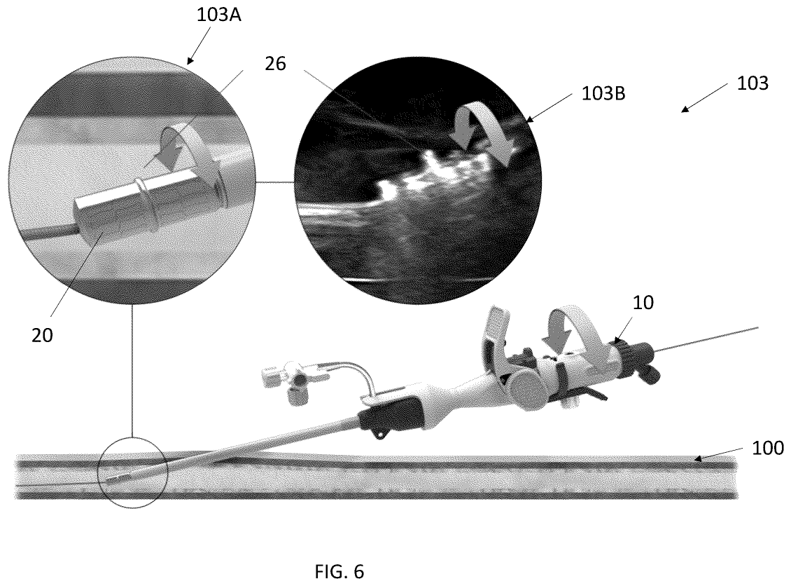

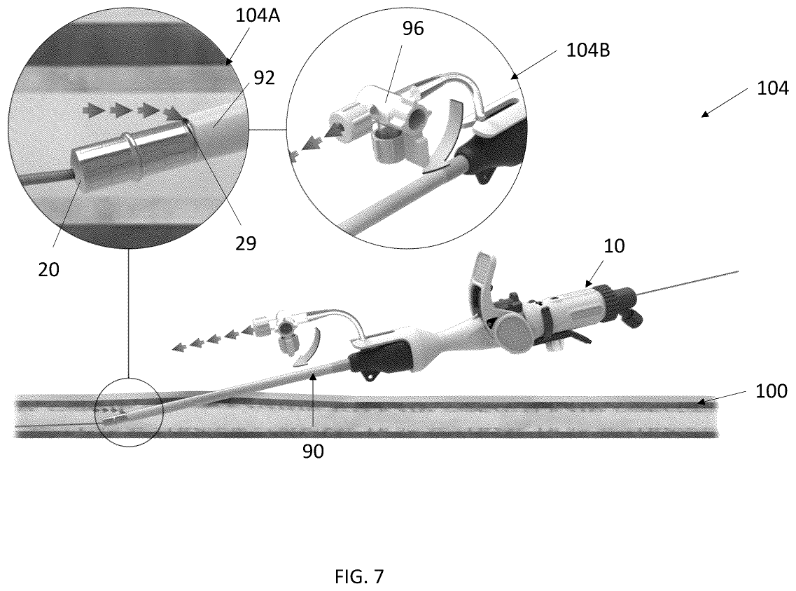

In this position, the undeployed coupler 20 can be viewed through imaging techniques, such as ultrasound, to confirm presence in the body lumen 100. For example, under ultrasound, the entire actuator 10 can be rotated about a longitudinal axis thereof to improve visibility. This is depicted in the viewing step 103 of FIG. 6, which includes an inset 103A depicting a roll motion of the coupler 20 and an inset 103B depicting an ultrasound image of the coupler 20 inserted into an actual body lumen 100. In both insets, the press ring 26 of the coupler 20 is especially visible. For further confirmation of the position of the coupler 20 in the body lumen 100, especially when the body lumen 100 is a vascular structure, the valve assembly 96 on the sheath 90 can be opened allowing blood to flow up the elongate shaft 92 of the sheath 90 in a gap defining a blood signal inlet 29 between the elongate shaft 92 and the coupler 20 as shown in the signal step 104 of FIG. 7. Blood can then exit via the valve assembly 96, thereby providing a simple visual confirmation that the coupler 20 is in proper position within the body lumen 100. Flow of the blood signal through the valve assembly 96 can be adjusted via manipulation of the valve assembly 96. The inset 104A depicts entry of blood into the gap 29 and the inset 104B depicts exit of blood out of the valve assembly 96.

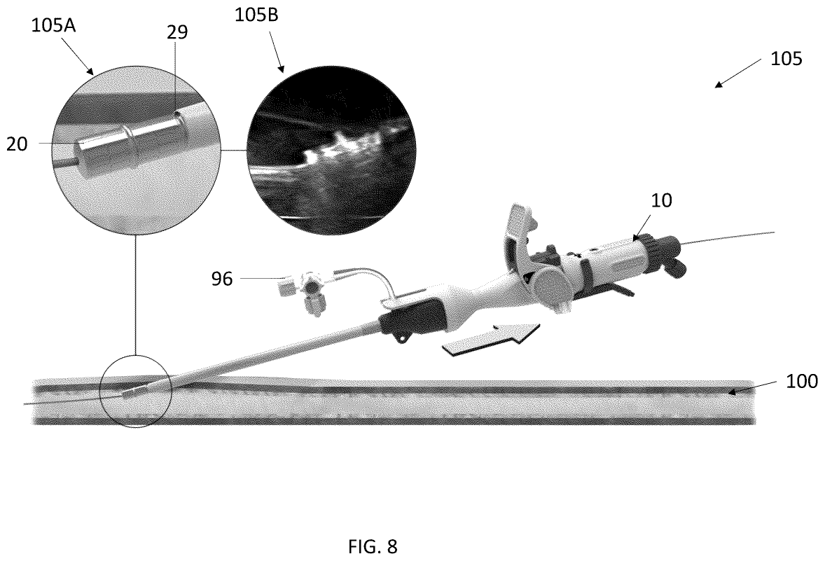

With the valve assembly 96 open, the actuator 10 can be pulled rearward until the blood signal stops. This cessation of the blood signal indicates that the gap defining the blood signal inlet 29 of the coupler 20 is located within a wall of the body lumen 100 rather than the body lumen 100 itself, as shown in the retraction step 105 of FIG. 8. The inset 105A depicts this in greater detail. Confirmation of this position can again be confirmed through medical imaging, such as ultrasound, and the inset 105B depicts such an ultrasound confirmation of the coupler 20 located within the wall of the body lumen 100.

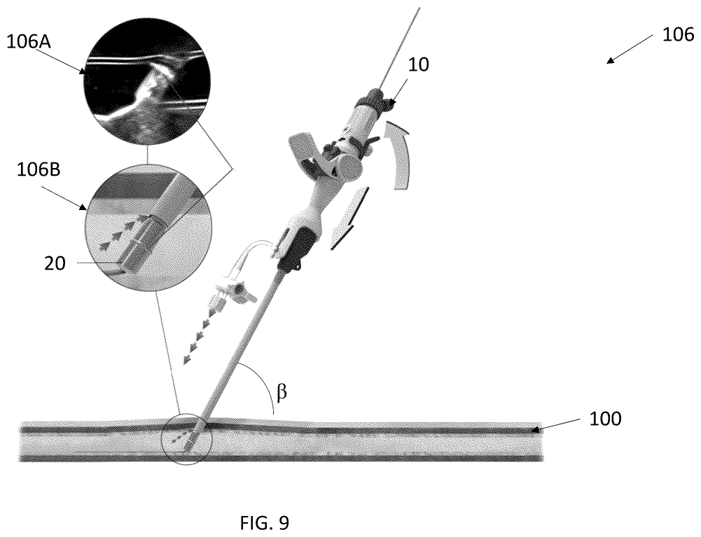

With the position confirmed, the entire actuator 10 can be elevated via an elevation angle β and maneuvered distally, seen in the elevation step 106 of FIG. 9. The elevation angle β can vary. For example, the elevation angle β can be between about 50 and 60 degrees relative to the body lumen 100. In this elevated and maneuvered position, the blood signal may resume. Confirmation of the position can be performed as described previously, and the insets 106A, 106B depict the coupler 20 in its new position in greater detail.

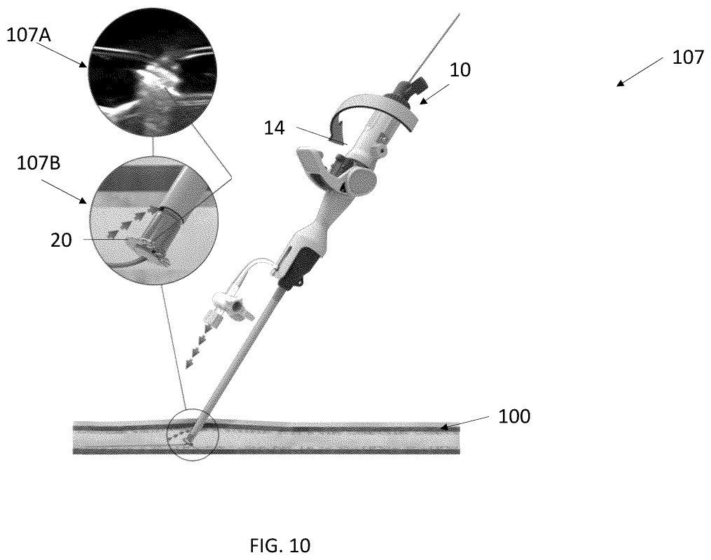

In the elevated position, the locking tab 14A can be removed from the actuator 10. Once the locking tab 14A is removed, the handle 14 can be manipulated relative to the rest of the actuator 10 to deploy the distal wing of the coupler 20 within the body lumen 100. This deployment is depicted in the deployment step 107 of FIG. 10. While clockwise rotation of the handle 14 is depicted, other manipulation types are possible as well, including the actuation of buttons, levers, etc., or through motions of other kind, including sliding, rotating, or manipulating in other ways. Deployment of the distal wing can be confirmed via the blood signal, as depicted, as well as via medical imaging as described herein. The insets 107A, 107B depict the coupler 20 with the distal wing deployed in the body lumen 100.

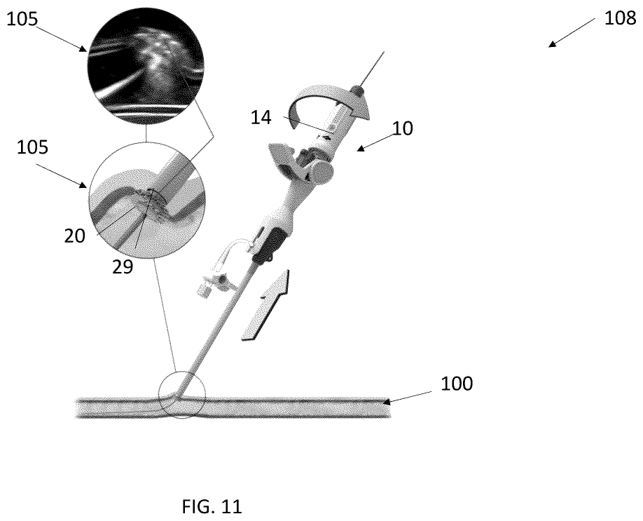

To deploy the proximal wing, the coupler 20 can be tensioned against the inner wall of the body lumen 100 by pulling proximally on the actuator 10 as shown in the deployment step 108 of FIG. 11. In this position, the gap 29 is located outside of the body lumen 100 ceasing the blood signal. The handle 14 can be actuated again to deploy the proximal wing of the coupler 20 outside the body lumen 100, thereby capturing tissue between the distal wing and proximal wing. Just as with deployment of the distal wing, the handle 14 can be actuated in a variety of ways. In the case of FIG. 11, the handle 14 is shown being rotated in a counter-clockwise direction. The inset 108A depicts a close-up view of the coupler 20 after deployment of the proximal wing and inset 108B represents an ultrasound view of the coupler 20.

More generally, a torsion force can be applied to the handle 14 in a first direction to deploy the distal wing and a second direction to deploy the proximal wing, the first direction being opposite the second direction. These torsional forces on the handle can be applied in isolation or in conjunction with a longitudinal force applied to the handle 14 in either the proximal or distal directions. The wings of the coupler 10 can be reversibly deployable so that, in the event of an improper deployment, a medical practitioner can correct the error. This will be described in greater detail below.

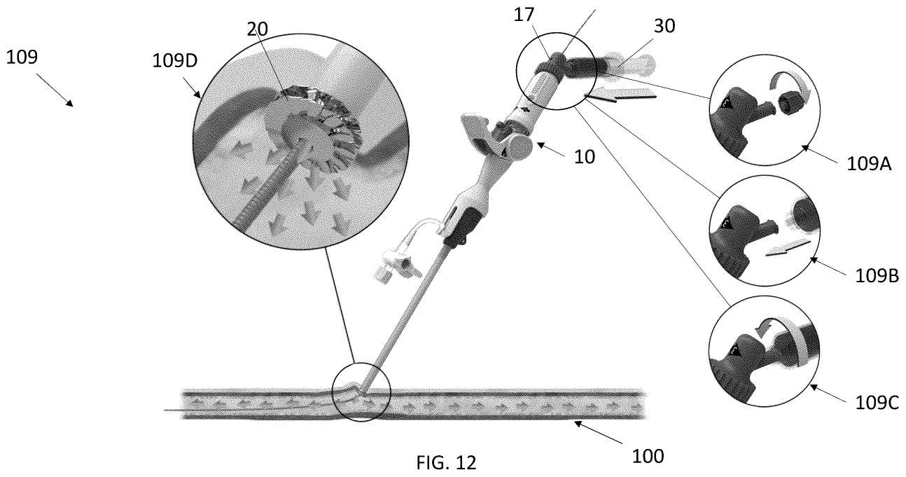

Confirmation of proper deployment can also be performed using contrast media. FIG. 12 depicts an example of a confirmation step 109. With the actuator 10 in the elevated position and following deployment of the distal and proximal wings, the contrast port 17 can be opened and a container 30 (e.g., a syringe) of contrast fluid can be coupled to the contrast port 17 as shown in insets, and contrast media can be introduced therethrough into the body lumen 100. Insets 109A-C respectively depict: the contrast port 17 being opened, the container 30 being introduced, and the container 30 being coupled to the contrast port 17. Inset 109D depicts contrast media flowing through the coupler 20 into the body lumen 100.

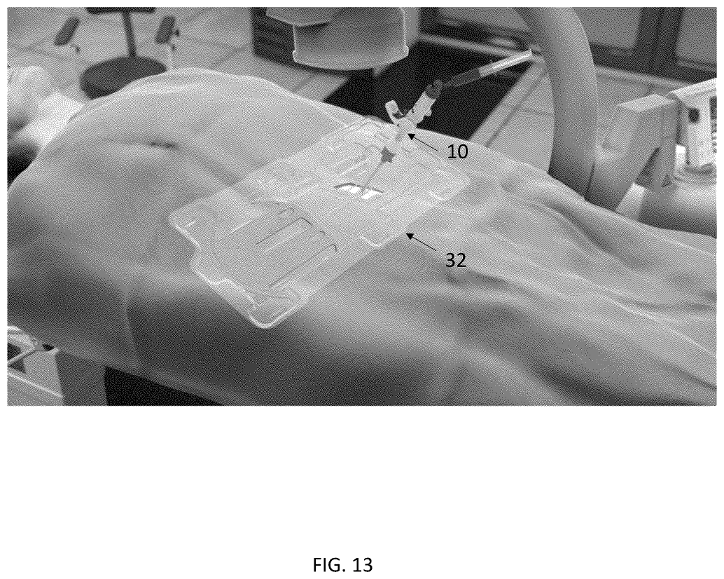

If desired at any step, the actuator 10 can be supported in a variety of ways, such as via a tray 32, shown in FIG. 13. This support tray 32 can also occur in conjunction with an imaging device, such as a C-arm or the like.

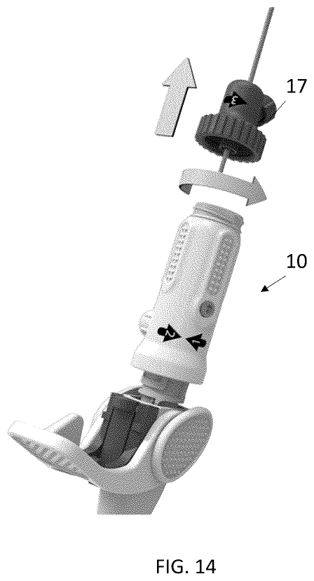

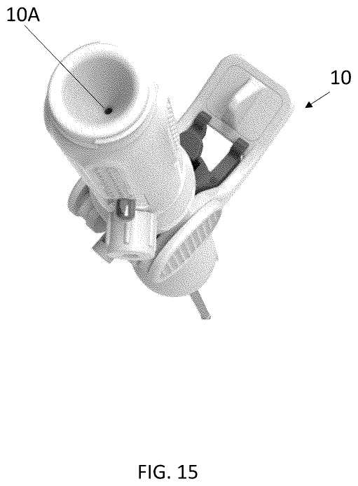

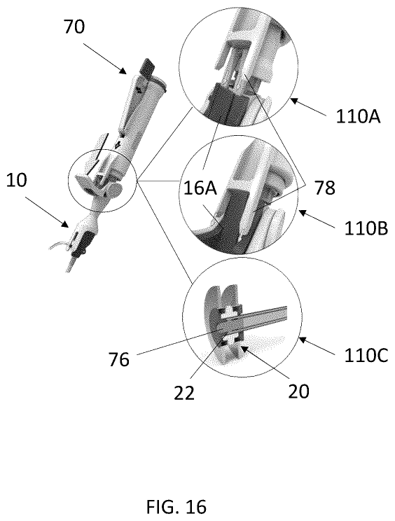

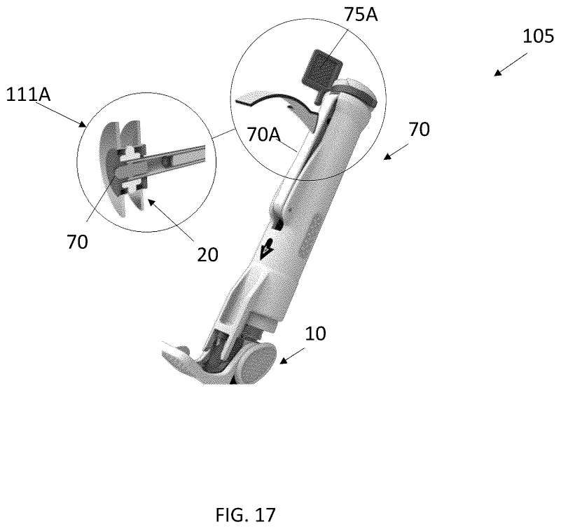

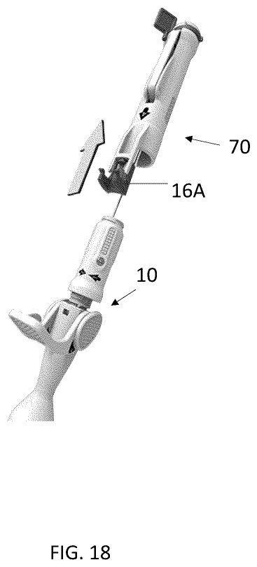

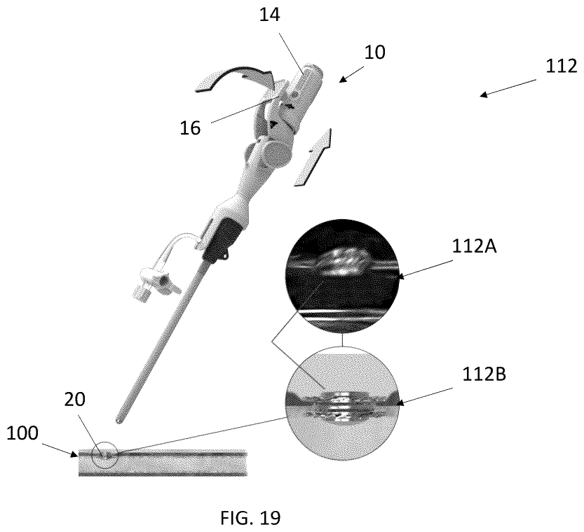

Once the wings are deployed, a plug can be delivered to the coupler 20 via the plug tool 70. As shown in FIG. 14, the contrast port 17 can be removed from the actuator 10 to expose the lumen 10A, seen especially in FIG. 15. The plug tool 70 can be inserted, plug 76 first, into the lumen 10A until the prongs 78 of the plug tool 70 couple to the lever lock 16A of the actuator 10, shown in the coupling step 110 of FIG. 16. This, in turn, advances the plug 76 into the coupler 20, plugging the lumen. Inset 110A depicts the prongs 78 sliding into position with the lever lock 16A, inset 110B depicts the prongs coupling to the lever lock 16A, and inset 110C depicts the plug 76 in position in the lumen 22 of the coupler 20. To deploy the plug 76, the plug tool lever lock 75A can be actuated to unlock the plug tool lever 70A. Once unlocked, the plug tool lever 70A can be pressed, causing the plug 76 to be ejected from the plug tool 70 into the coupler 20. This ejection process 111 is depicted in FIG. 17, with the inset 111A depicting the plug 76 detached from the plug tool 70. After the plug 76 has been ejected, the plug tool 70 can be withdrawn from engagement with the actuator 10, as shown in FIG. 18. This action results in removal of the lever lock 16A from engagement with the actuator 10. After ejecting the plug 76, the coupler 20 can be deployed and the actuator 10 removed from the body lumen 100. By actuating the ejection lever 16 until it abuts the handle 14, the coupler 20 can be detached from engagement with the actuator 10 as shown in the deployment step 112 of FIG. 19. This deployment can be verified using medical imaging, such as ultrasound, as shown in the insets 112A, 112B of FIG. 19.

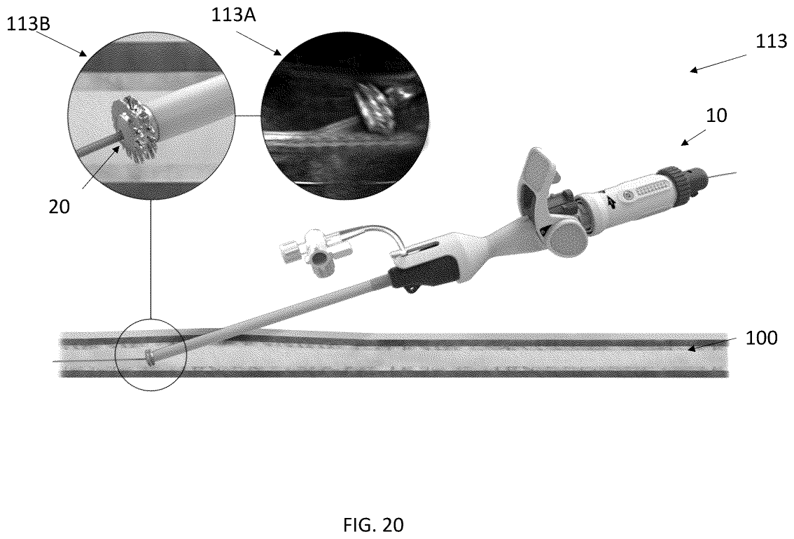

If the proximal and distal wings of the coupler 20 are improperly deployed within the body lumen 100, the procedure of FIGS. 20-26 can be followed to remove the coupler 20 from the body lumen 100. This improper deployment 113 is depicted especially in FIG. 20, and the insets 113A, 113B depict the coupler 20 in an improperly deployed state within the body lumen 100. Both the proximal and distal wings of the coupler 20 are deployed within the body lumen 100, rather than being deployed to couple tissue. If the coupler 20 is ejected in this state, the coupler will be free-floating in the body lumen 100, which could lead to harmful consequences for a patient. At the same time, the coupler 20 is now in a deployed state and cannot be merely removed from the body lumen 100 without causing additional patient harm. The removal process of the ensuing figures addresses the manner in which a medical professional may safely remove an improperly deployed coupler 20.

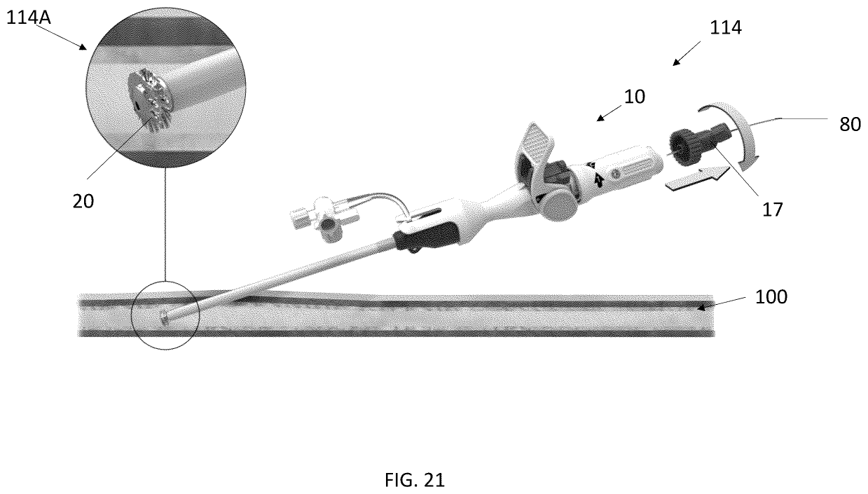

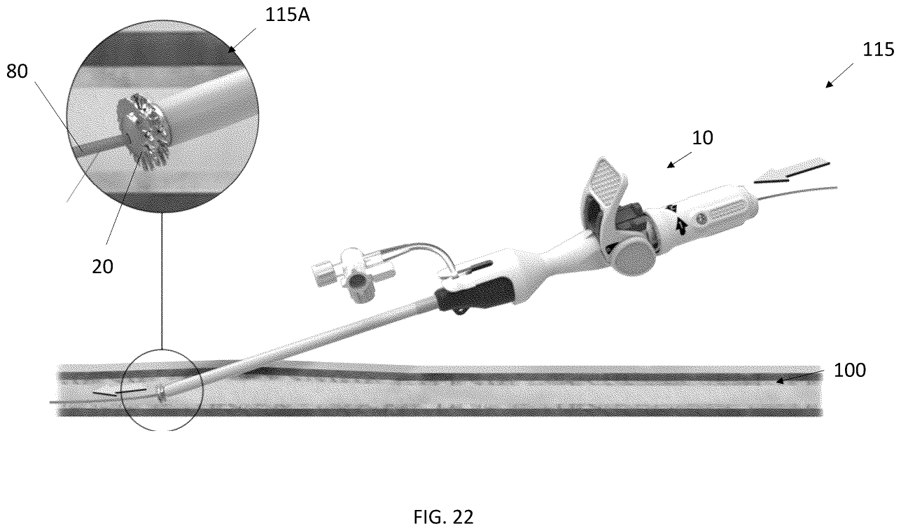

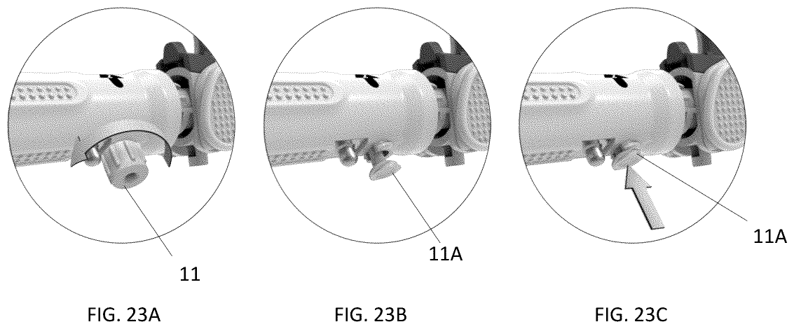

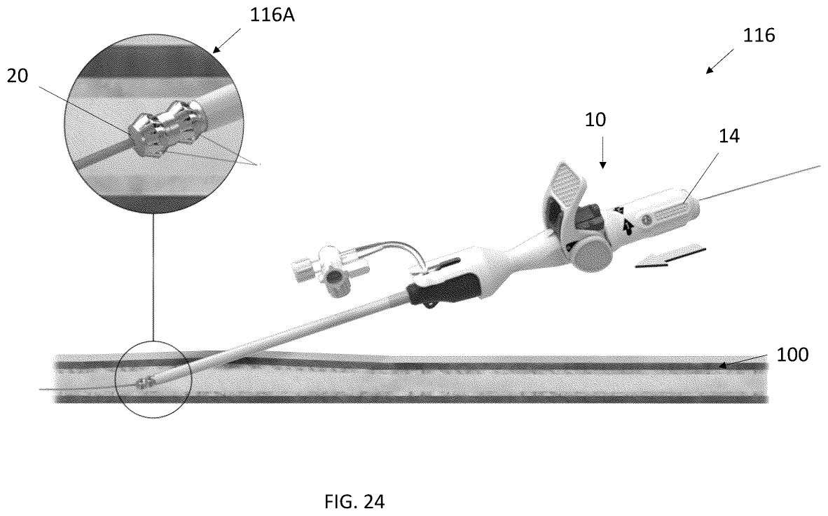

FIG. 21 depicts the first step 114, which involves removal of the contrast port 17 and guidewire 80 from the lumen 10A of the actuator 10. This removal can be seen especially in the inset 114A, which depicts the guidewire 80 no longer protruding from the coupler 20. A replacement guidewire 80, such as a super or ultra stiff guidewire, can be inserted through the actuator 10 and into the body lumen 100 as shown in the insertion step 115 of FIG. 22. The inset 115A depicts the new guidewire 80 extending past the improperly deployed coupler 20. Once the new guidewire 80 is in position, a cap 11 located on the actuator 10 can be opened, and a spring release mechanism 11A can be actuated to allow the deployed wings of the coupler 20 to collapse. This removal and actuation can be seen in FIGS. 23A-C. By moving the handle 14 of the actuator 10 in a distal direction, the coupler 20 can fully collapse to the state depicted in the collapsing step 116 of FIG. 24, seen especially in the inset 116A.

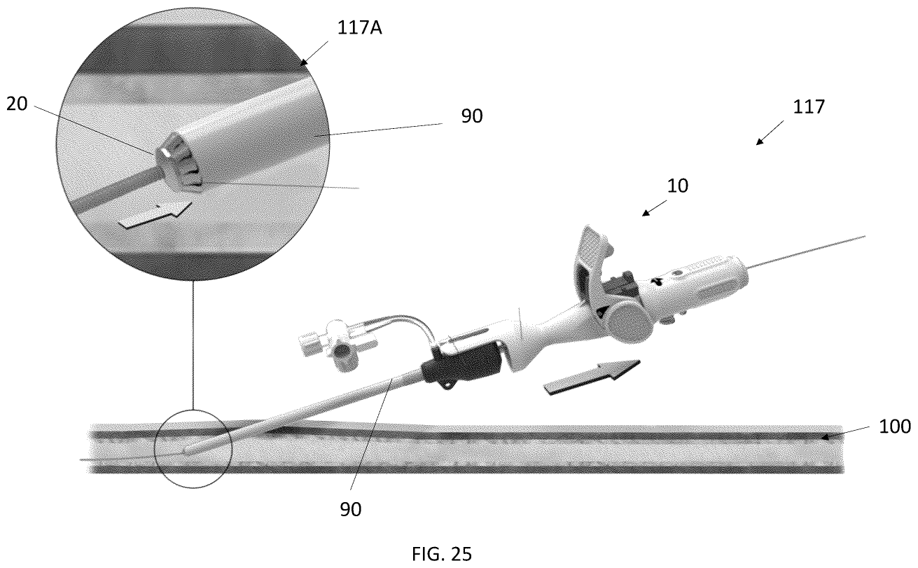

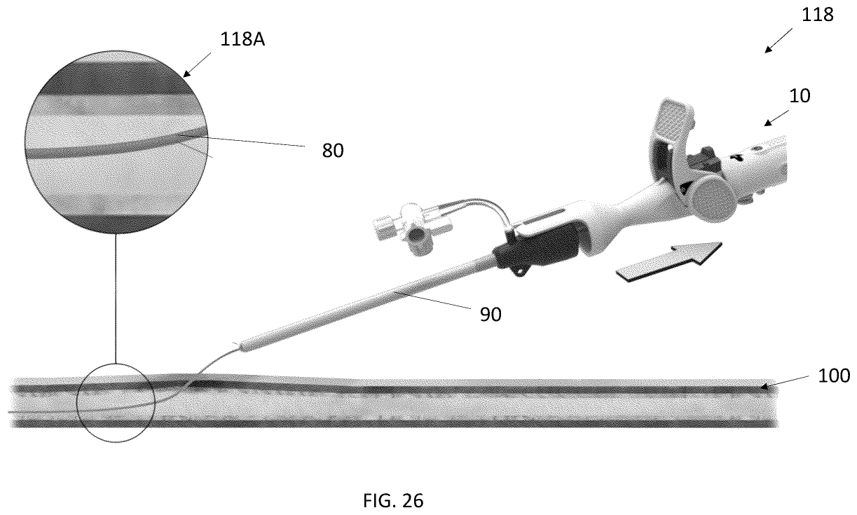

After the coupler 20 has collapsed, the actuator 10 can be moved proximally to disengage the sheath 90, drawing the coupler 20 into the lumen of the sheath 90 at least partially as shown in the withdrawal step 117 of FIG. 25, and especially in the inset 117A. FIG. 26 depicts a retraction step 118 in which the actuator 10 and the sheath being withdrawn from the body lumen 100 over the new guidewire 80, seen in the inset 118A. With the new guidewire 80 in place, a new introducer sheath 90 can be inserted, and the process can begin again with a new actuator 10 and coupler 20.

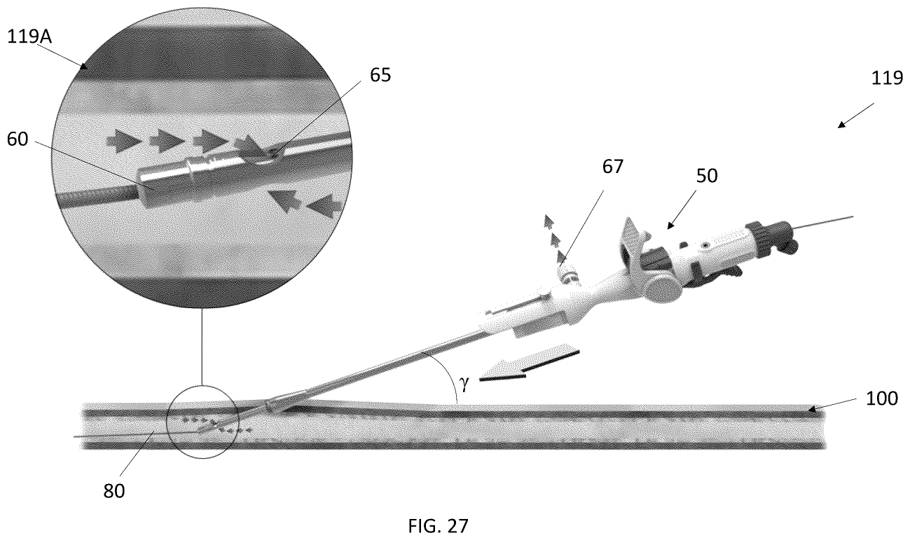

FIGS. 27-34 depict a procedure used with the second variation of actuator 50 described above. Many of the steps in the procedure are similar to those of the procedure described with respect to the first variation of actuator. For brevity, individual steps of this second procedure will not be described in great detail when a direct comparison to the first procedure will suffice. As applied to the procedure for the first variation, the procedure for the second variation can be confirmed via medical imaging, such as ultrasound, in a manner similar to that described above. Insets to FIGS. 27-34 depicting this imaging are not shown, but the results can be the same.

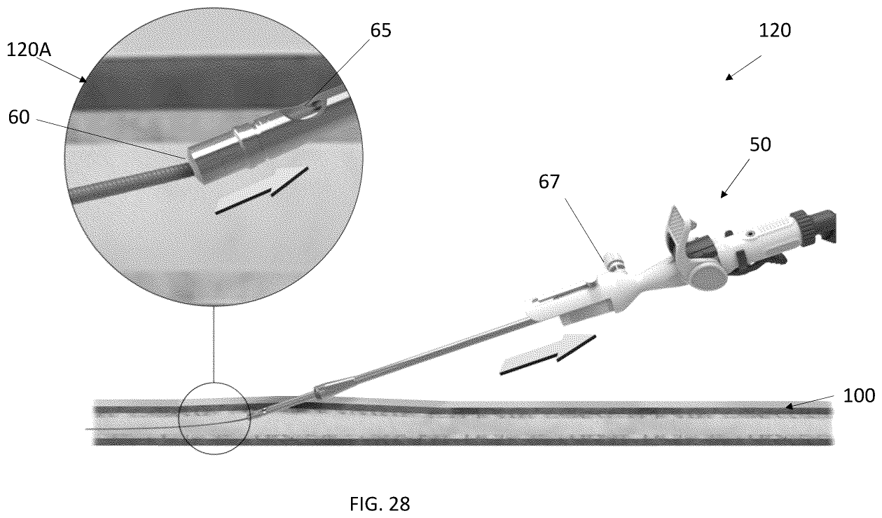

As introduced above, the second variation procedure details the deployment of a coupler 60 having a distal wing and a slide plate 55 rather than a distal wing and a proximal wing. Generally, the actuator 50 can be introduced to a body lumen 100 over a guidewire 80 in a substantially similar manner as the first variation actuator 10. The actuator 50 is introduced at a similar angle of approach γ, and once in position, a blood signal outlet 67 on the actuator can be opened to confirm proper positioning of the coupler 60 within the body lumen 100 as depicted in the introduction step 119 of FIG. 27. Blood entering a blood signal inlet 65 is depicted in the inset 119A. The blood can flow through the actuator 50 and exit out the blood signal outlet 67. Once proper positioning is configured confirmed, the actuator 50 can be withdrawn as shown in the withdrawal step 120 of FIG. 28 until the blood signal leaving the outlet 67 disappears, indicating that the blood signal inlet 65 is in the proper position within the tissue for deployment of the distal wing of the coupler 60 to take place within the body lumen 100. This position is depicted in the inset 120A.

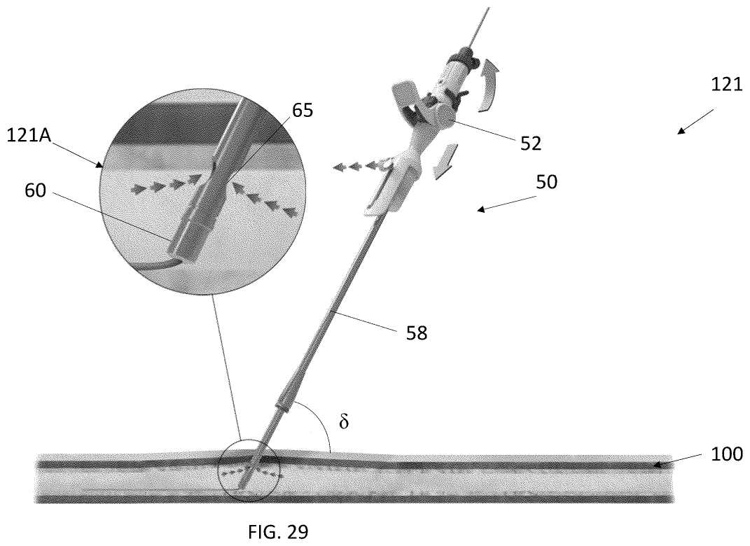

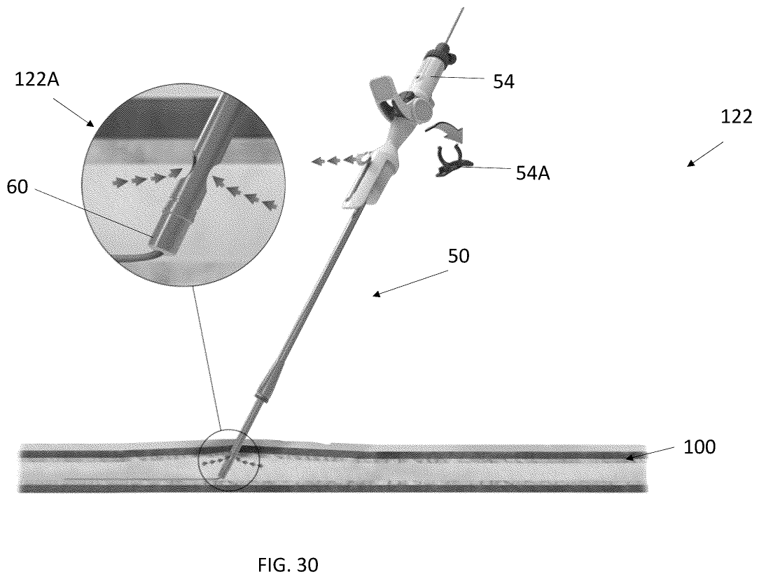

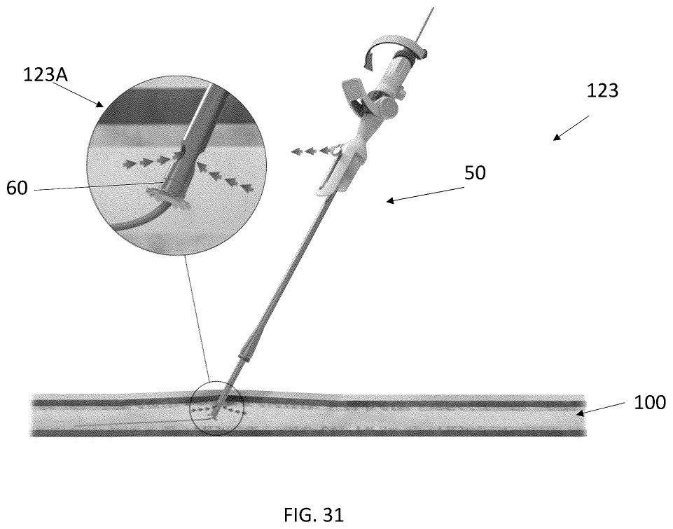

With respect to the signaling step 121 of FIG. 29, the actuator 50 can be elevated at an elevation angle δ relative to the body lumen 100, and the actuator 50 can be moved distally such that the blood signal returns. The inset 121A depicts the blood signal inlet 65 in greater detail. To deploy the distal wing, the locking tab 14A can be removed from the handle 14, as shown in the preparation step 122 of FIG. 30, and the handle 14 can be actuated to deploy the distal wing within the body lumen 100 as shown in the deployment step 123 of FIG. 31. All the while, the blood signal should continue to flow, indicating that the coupler 20 remains in its proper position within the body lumen 100. The inlet 123A of FIG. 31 depicts the blood signal continuing to flow.

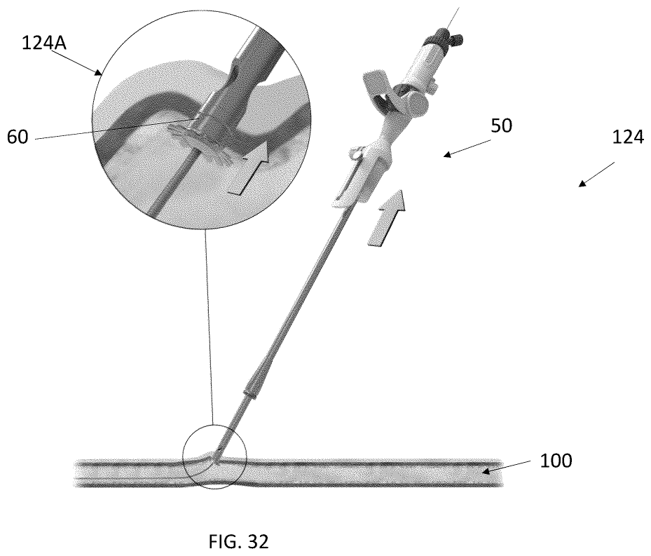

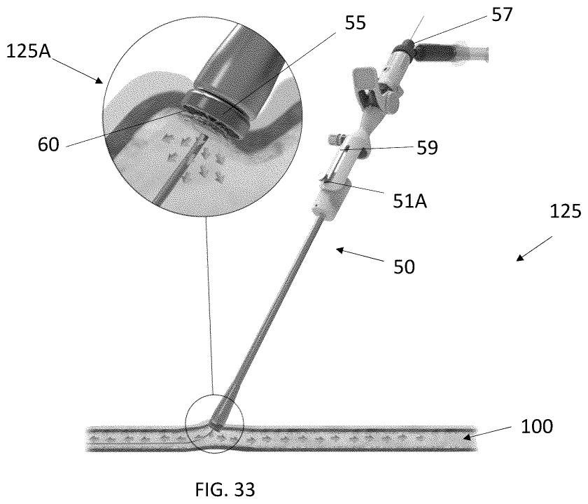

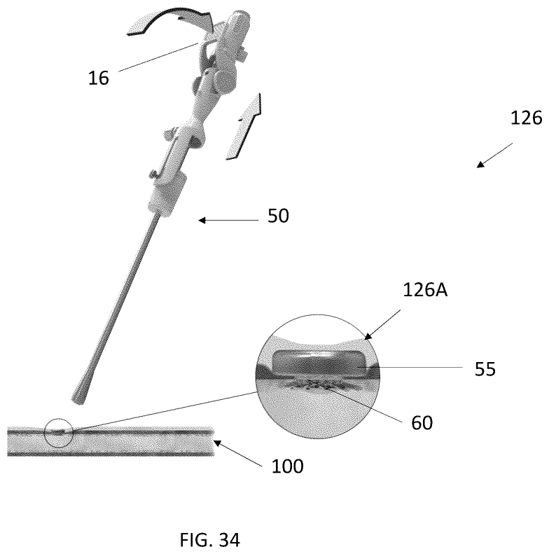

Once the distal wing is deployed, the actuator can be moved proximally to tension the coupler 20 against an interior wall of the body lumen 100 as shown in the tensioning step 124 of FIG. 32. Tensioning the actuator 50 in this manner positions the blood signal inlet 65 outside of the body lumen 100, and the blood signal ceases. This positioning can be seen in greater detail in the inset 124A. Next, the slide plate 55 of the actuator 50 can be advanced by sliding the guide pin 51A in the track 59 of the actuator 50 to advance the outer sheath 58 in a distal direction and the slide plate 55 with it. The slide plate 55 can be advanced until it contacts and couples with the coupler 20 as depicted in FIG. 33. As explained above, contrast fluid can be deployed via the contrast port 57 on the actuator 50 as shown in FIG. 33. The inset 125A depicts contrast fluid flowing into the body lumen 100. Ejection of the coupler 60 with the slide plate 55 is depicted in the ejection step 126 of FIG. 34 Actuation of the ejection lever 16 causes the coupler to be ejected, and the actuator 50 can removed. The ejected coupler 60 with slide plate 55 is depicted in the inset 126A in greater detail.

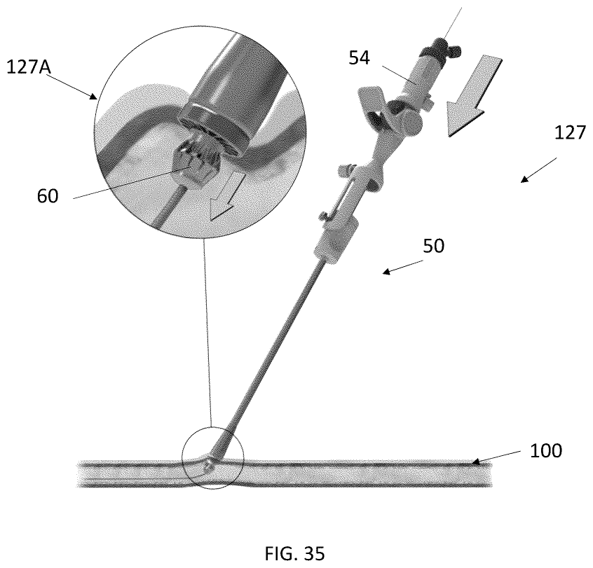

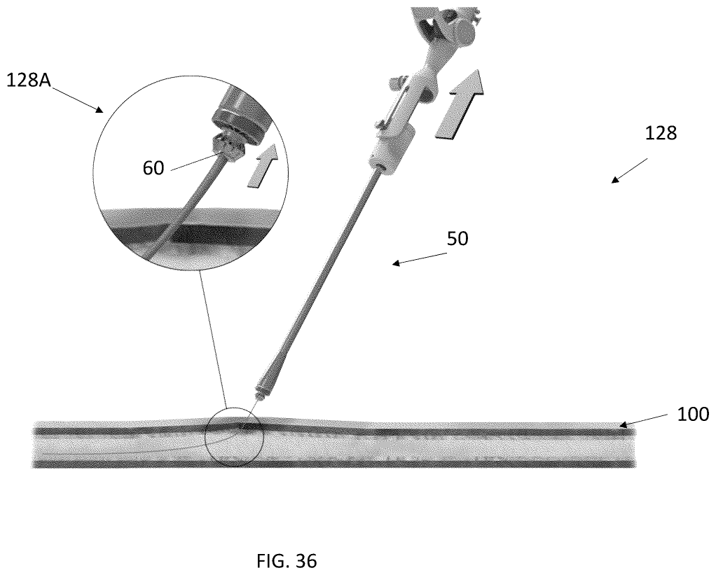

If the coupler 20 is improperly deployed, procedures depicted in FIGS. 35-36 can be followed to safely remove the actuator and coupler. These procedures are similar to those described above with respect to FIGS. 20-26. A cap (not shown) on the actuator 50, similar to that shown in FIG. 23, can be removed and a spring release mechanism (not shown) can be actuated in conjunction with distal movement of the handle 54, shown in the collapsing step 127 of FIG. 35, to collapse the distal wing of the coupler 60. The inset 127A depicts the collapsed actuator 60. Once collapsed, the actuator 60 can be withdrawn over the guidewire 80 from the body lumen 100 as seen in FIG. 36. The inset 128A depicts this removal. A new actuator 60 can be introduced and the procedure can begin again from the beginning.

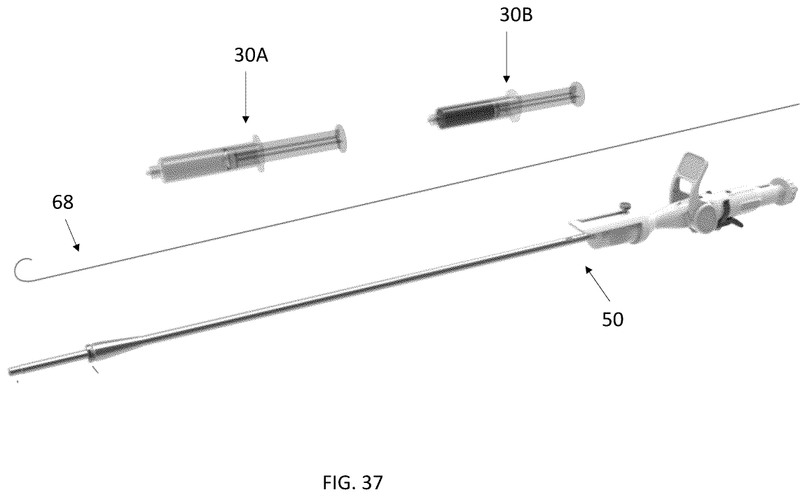

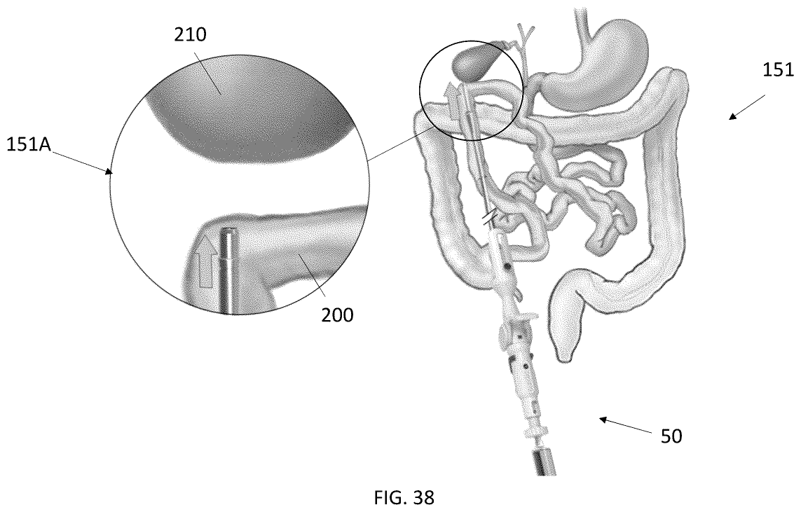

The promotion of weight loss and/or treatment of Type II diabetes can be effected with a cholecystoileostomy (bile bridge) or cholescystojejunostomy and an optional entero-entero anastomosis using a specially-designed medical instrument and accompany procedural process. Generally, an implant is deployed within a patient's body to: 1) provide an anchor, via the distal disk, within the gallbladder 210; 2) provide a lumen through which bile can flow from the gallbladder 210 to the bowel 200; and 3) facilitate the docking of a slide plate portion to the implant body. An exemplary instrument set is detailed in FIG. 37, and an exemplary process is detailed in FIGS. 38-53. Each will be described in turn.

FIG. 37 depicts instruments that may be used in the exemplary process detailed in FIGS. 38-53, including a saline syringe 30A, a contrast syringe 30B (such as methylene blue), a guard wire 68 (optional), and a bile-bridge delivery system in the form of actuator 50.

Generally, in operation, a distal portion of the actuator 50 is inserted via laparoscopic means into the bowel 200 of a patient, and it is advanced to a portion of the bowel 200 proximate the gallbladder 210. This portion of the bowel 200 will be the site of the anastomosis. The penetrator element 63 of the delivery system, as seen in FIG. 2B, can be exposed by depression of a penetrator knob on the actuator 50, and the actuator 50 can be advanced via the penetrator 63 though the bowel 200 and gallbladder 210 walls. Once inserted, the coupler 60 can be deployed to expand first a distal wing, and the slide plate 55 can be advanced toward the coupler 60 to lock the elements together with tissue therebetween. The coupler can be ejected and deployed within the patient.

In the event that either the distal wing of the coupler 60 is incorrectly deployed or if a surgeon is unsatisfied with the quality of placement, a cap on the actuator 50 can be removed to reveal an actuator button, when actuated, will allow the collapse of the coupler 60 and permit removal of the actuator 50. Similar procedures are described above.

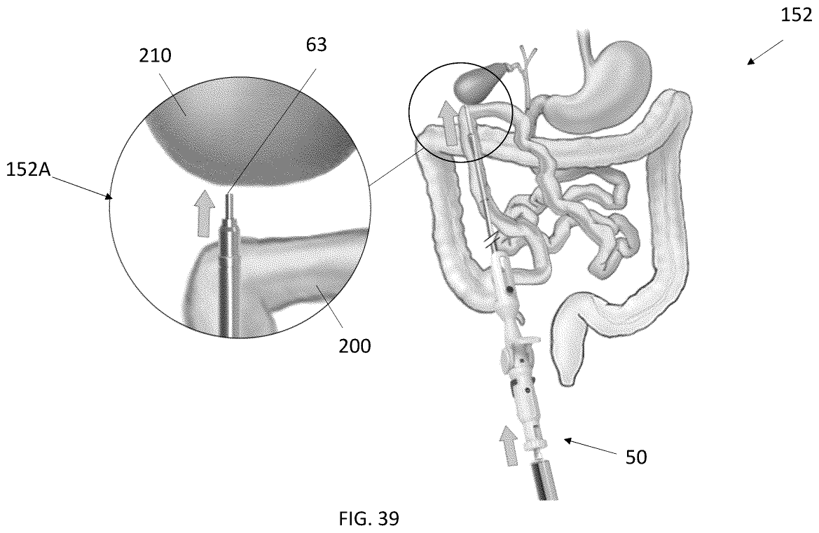

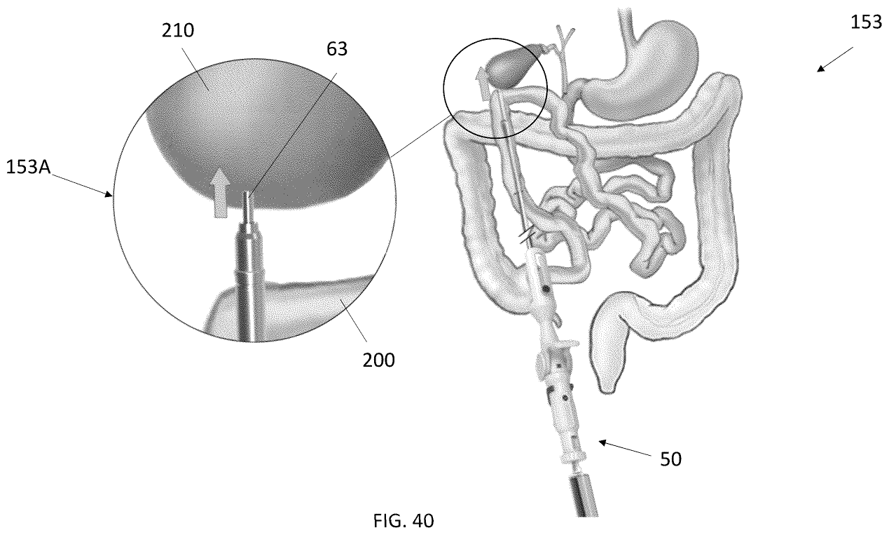

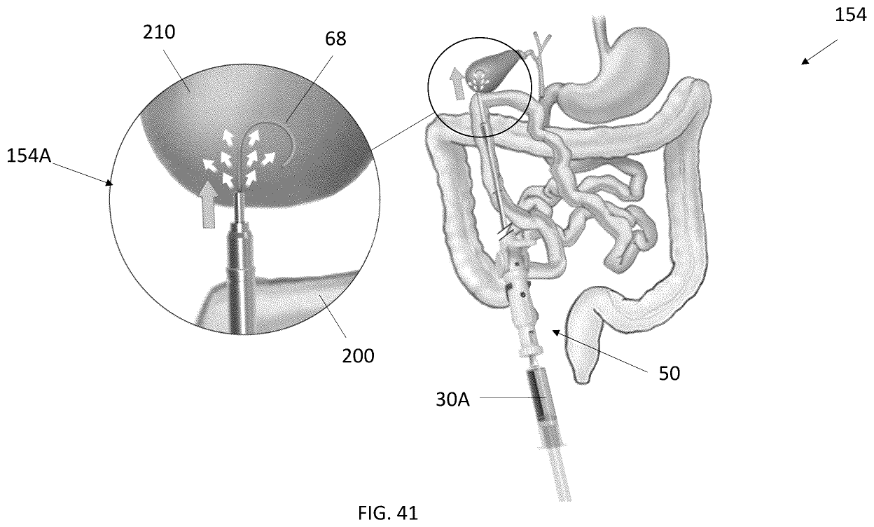

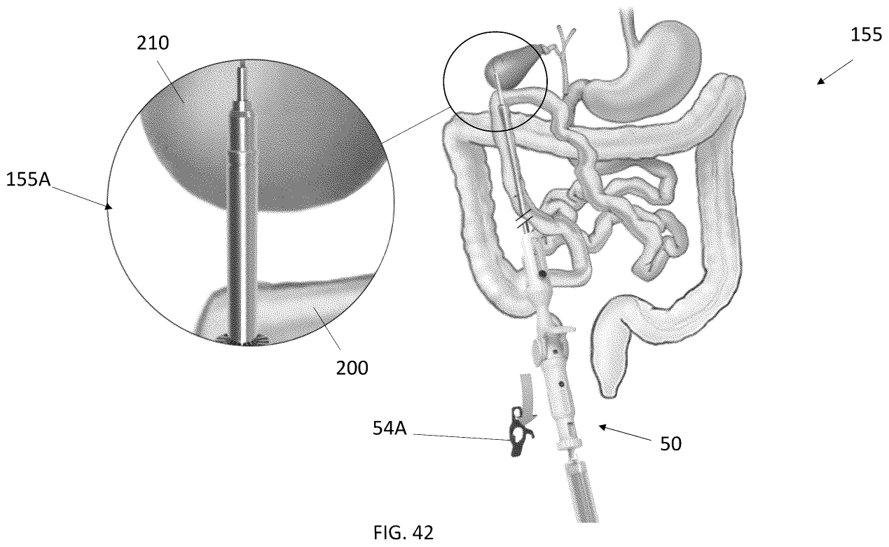

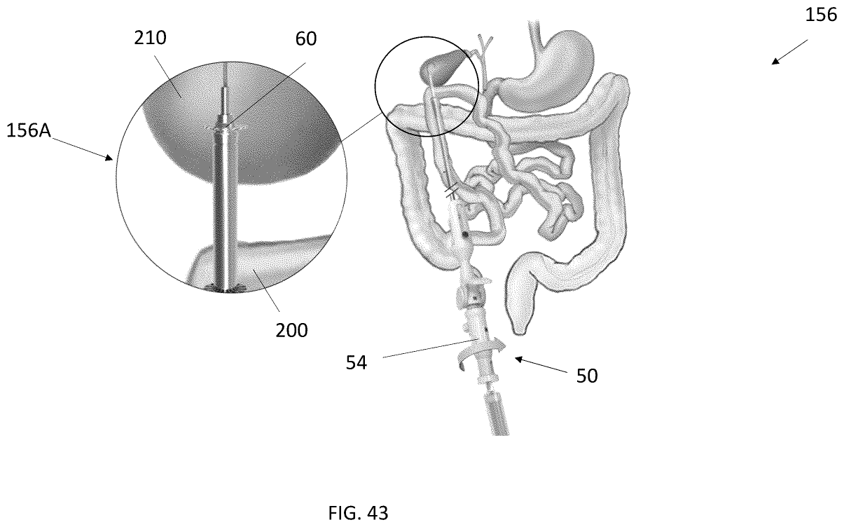

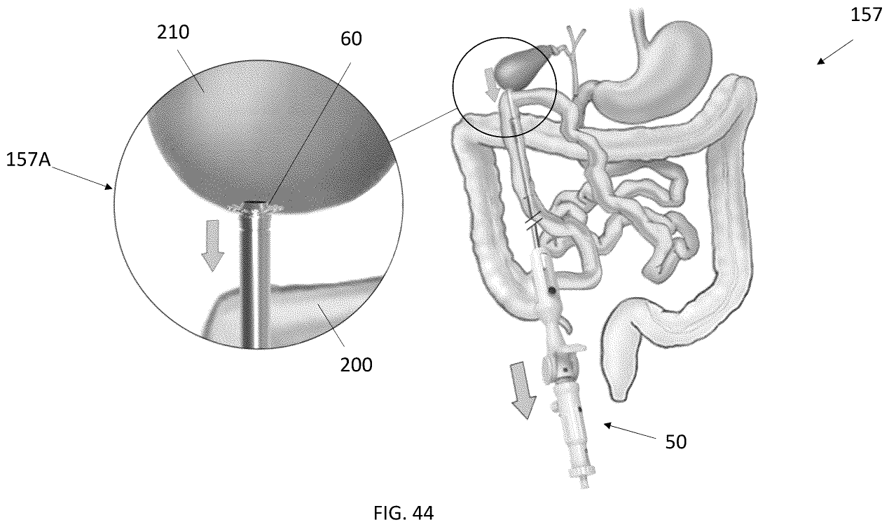

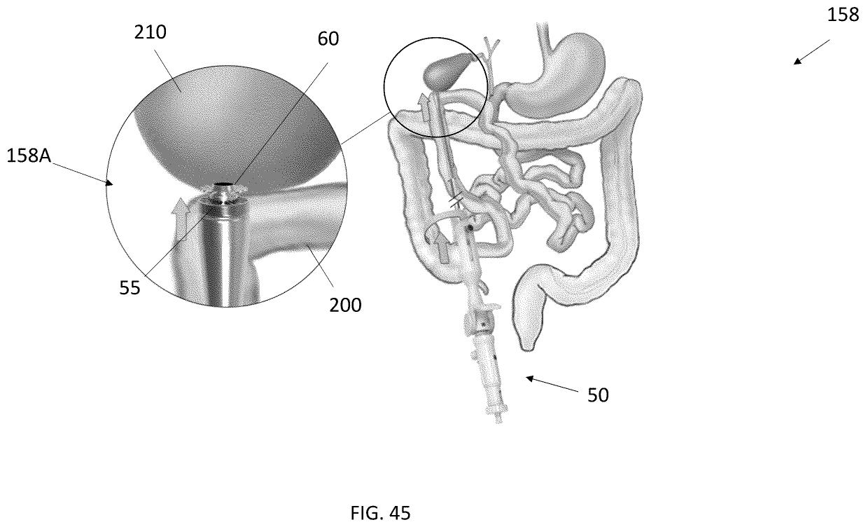

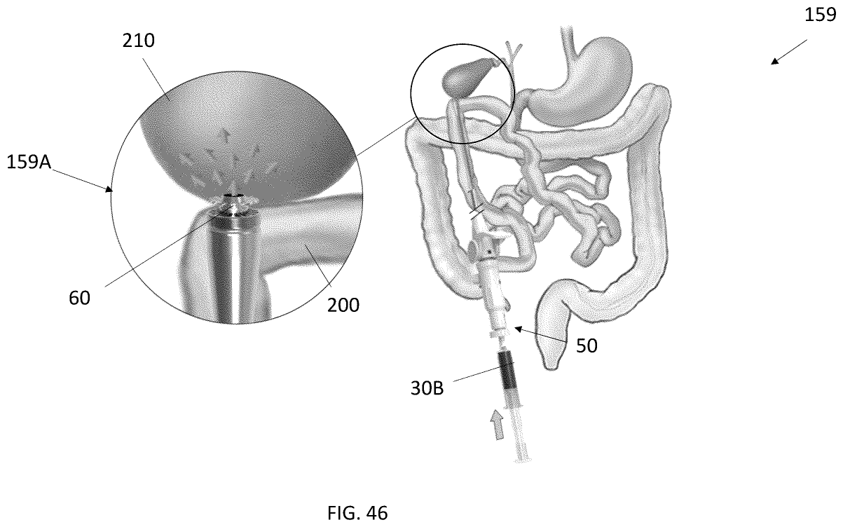

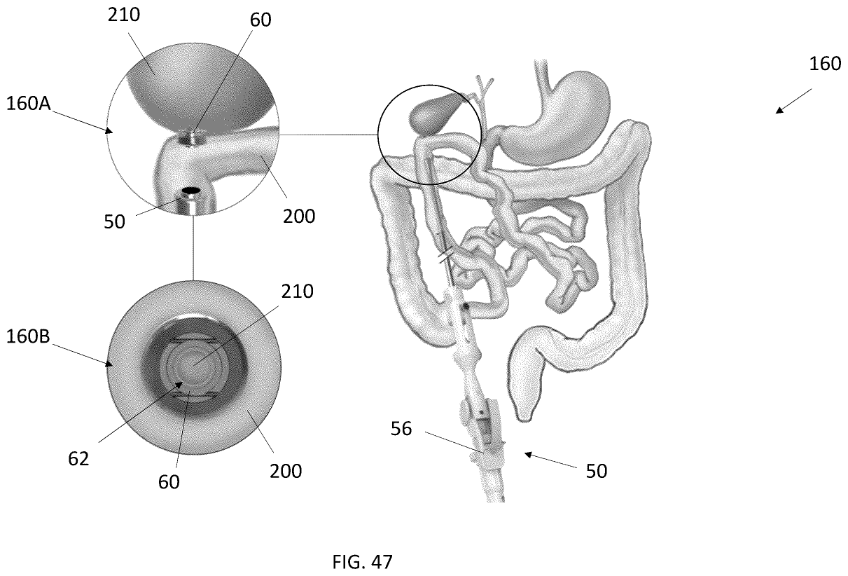

Turning now to the exemplary procedure, a cholecystoileostomy and an entero-entero anastomosis depicted in FIGS. 38-53, the first step consists of selecting the ileum loop of the bowel 200 and bringing it to an antecolic or retro-colic position. A surgeon can also select and move a portion of a patient's jejunum. A 5 mm antimesenteric enterotomy can be performed while the ileum loop remains in the antecolic position (or a retrocolic position—not shown). The actuator 50 of FIG. 37 can be inserted into the enterotomy until it is positioned against a wall of the ileum proximate the gallbladder 210 as seen in the placement step 151 of FIG. 38. The inset 151A depicts the placement in finer detail. Using the penetrator 63, the wall of the ileum can be penetrated, ileal penetration step 152 of FIG. 39, and penetration of the gallbladder 210, gallbladder penetrations step 153 of FIG. 40, can follow. FIGS. 39 and 40 include respective insets 152A, 153A, which depict these penetration steps 152, 153 in detail. The penetrator 63 can be a cutting needle, RF probe, circular punch, or equivalent known in the art. Once the tip of the actuator 50 is disposed in the gallbladder 210, the saline syringe 30A can be flushed through the actuator 50 to distend the gallbladder 210. In one version, this saline flush via saline syringe 30A can also advance the guardwire 68 into the gallbladder 210 as seen in the advancement step 154 of FIG. 41. The inset 154A depicts saline entering the gallbladder 210 around the advanced guardwire 68 in greater detail. Once in position, the locking tab 54A can be removed, removal step 155 of FIG. 42, and the handle 54 of the actuator 50 can be actuated to deploy the distal wing of the coupler 60 within the interior of the gallbladder 210, deployment step 156 of FIG. 43. The inset 155A of FIG. 42 depicts the position of the advanced coupler 60 within the gallbladder 210 pre-deployment, while the inset 156A of FIG. 43 depicts the advanced coupler 60 with the distal wing deployed. With the distal wing deployed, the actuator 50 can be drawn proximally against the interior of the gallbladder 210 to move the gallbladder 210 proximate the punctured small bowel 200, as seen in the retraction step 157 of FIG. 44. The inset 157A depicts the distal wing of the coupler 60 contacting an interior of the gallbladder 210. The slide plate 55 can now be advanced distally to join the slide plate to the coupler with the gallbladder 210 and small bowel 200 captured therebetween, as seen in the coupling step 158 of FIG. 45. The inset 158A depicts the coupled coupler 60 and slide plate 55. Contrast fluid, such as methylene blue via the contrast syringe 30B, can be injected via the lumen of the actuator 50 to confirm that no leakage of the anastomosis has occurred, depicted in the confirmation step 159 of FIG. 46. The inset 159A depicts the contrast fluid entering the gallbladder 210. The ejection handle 56 of the actuator 50 can be actuated to deploy the coupler 60 and slide plate 55 as shown in the ejection step 160 of FIG. 47. The inset 160A depicts the disconnected actuator 50, and the inset 160B depicts a view of the gallbladder 210 from the bowel 200 via the central lumen 62 of the coupler 60.

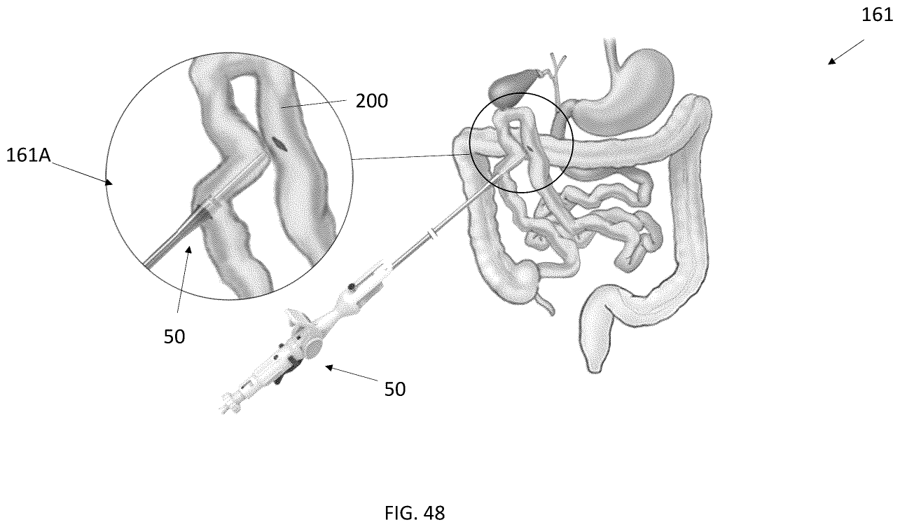

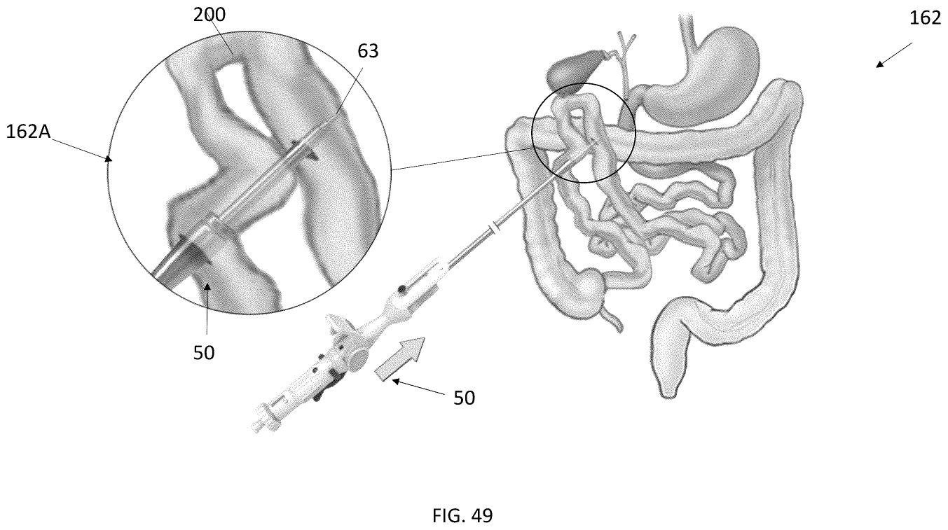

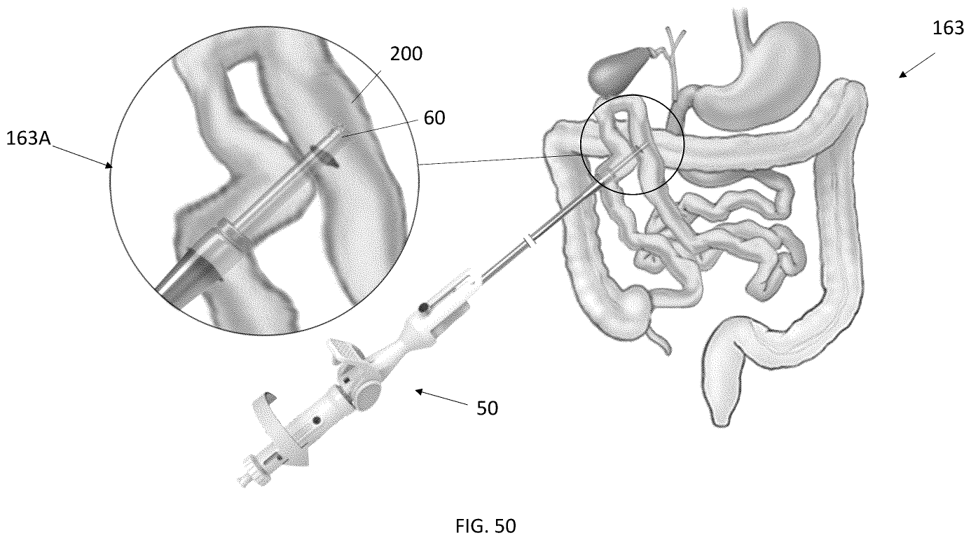

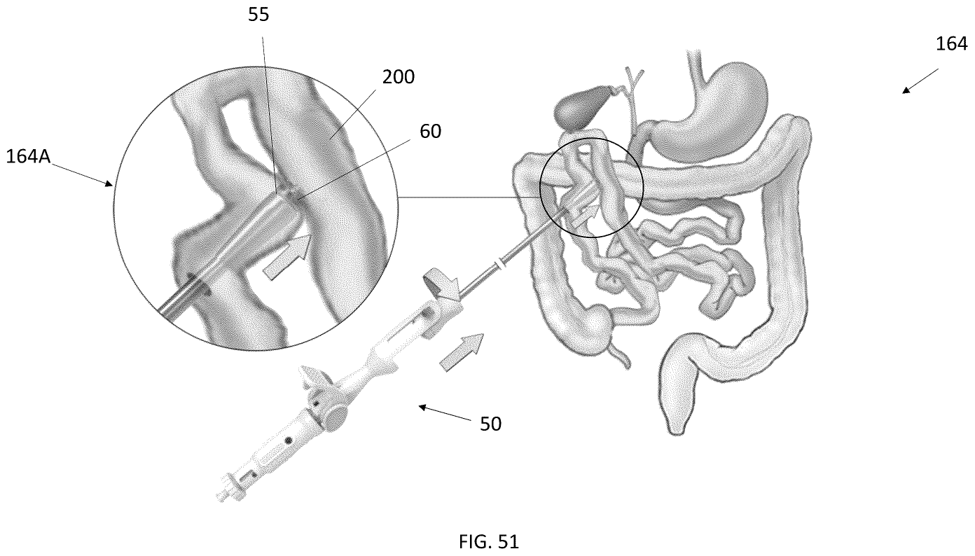

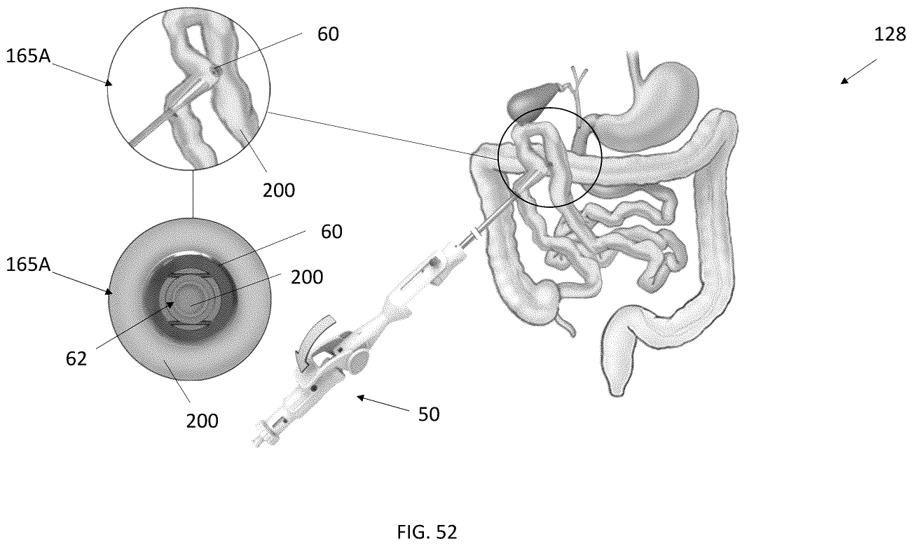

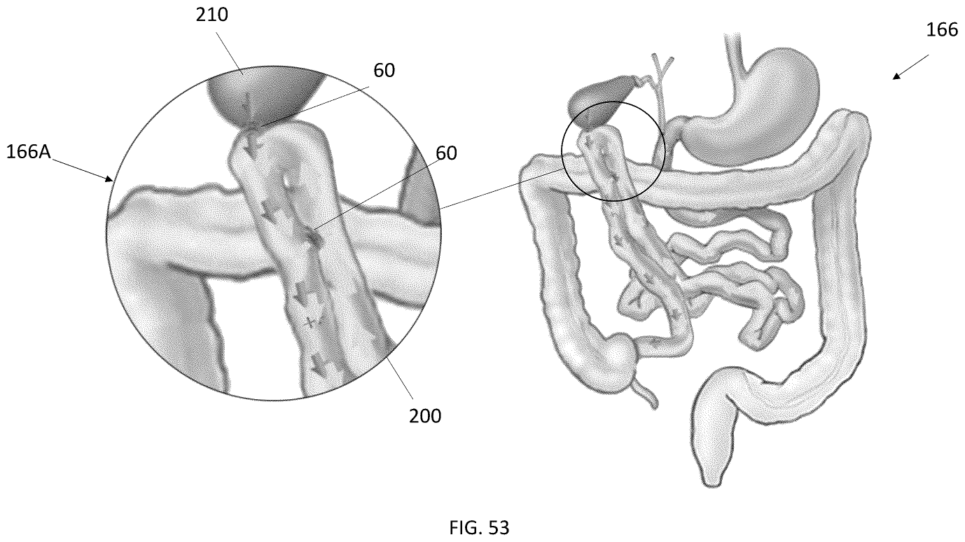

With the first coupler 60 placed, the actuated actuator 50 can be fully withdrawn, and a new actuator 50 can be inserted into the enterotomy. A loop of the ileum proximal to the deployed coupler can be brought close to the new actuator 50. A second 5 mm enterotomy can be performed in the proximal ileal loop, the actuator 50 can be aligned with the newly-created enterotomy, seen in the alignment step 161 of FIG. 48, and the actuator 50 can be advanced via a penetrator 63 through the wall of the distal ileal and into the proximal ileal loop via the enterotomy, seen in the advancement step 162 of FIG. 49. The inset 161A of FIG. 48 depicts the alignment in greater detail, and the inset 162A depicts the advancement in greater detail. Once in position, the locking tab 54A of the second actuator 50 can be removed and, while the coupler 60 is located in the proximal ileal loop, the handle 54 can be actuated to deploy the distal wing of the coupler 60 therein, seen in the actuation step 163 of FIG. 50. The actuator 50 can be withdrawn to tension the proximal ileal loop against the distal ileal loop and the slide plate 55 can be advanced and locked to the coupler 60 as shown in FIG. 51. The ejection handle 56 can be deployed to eject the coupler 60 as shown in FIG. 52, and the actuator 50 can be fully withdrawn. The enterotomy can be closed with a suture. FIG. 53 depicts bile flow from the gallbladder 210 through the small intestine and the entero-entero anastomosis as well as bile flow from the gallbladder 210 through the cholecystoileostomy loop via the placed couplers 60.

Certain illustrative implementations have been described to provide an overall understanding of the principles of the structure, function, manufacture, and use of the systems, devices, and methods disclosed herein. One or more examples of these implementations have been illustrated in the accompanying drawings. Those skilled in the art will understand that the systems, devices, and methods specifically described herein and illustrated in the accompanying drawings are non-limiting illustrative implementations and that the scope of the present invention is defined solely by the claims. The features illustrated or described in connection with one illustrative implementation may be combined with the features of other implementations. Such modifications and variations are intended to be included within the scope of the present invention. Further, in the present disclosure, like-named components of the implementations generally have similar features, and thus within a particular implementation each feature of each like-named component is not necessarily fully elaborated upon.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

One skilled in the art will appreciate further features and advantages of the invention based on the above-described implementations. Accordingly, the present application is not to be limited by what has been particularly shown and described, except as indicated by the appended claims. All publications and references cited herein are expressly incorporated by reference in their entirety.

What is claimed is:

1. A surgical assembly, comprising:
an introducer sheath defining a central lumen, the introducer sheath including a blood signal port located at a proximal end thereof;
an actuator having an elongate shaft extending distally therefrom, the actuator being configured to couple to the introducer sheath at the proximal end such that the elongate shaft extends through the central lumen of the introducer sheath; and
a deployable coupler coupled to a distal end of the elongate shaft, the deployable coupler having a plurality of proximal slits configured to form a proximal wing and a plurality of distal slits configured to form a distal wing, the deployable coupler being disposed distally of the introducer sheath when the actuator is coupled to the introducer sheath;
wherein a blood signal flow inlet is defined between the introducer sheath and the deployable coupler when introducer sheath and the actuator are coupled together such that blood can enter the blood signal flow inlet disposed within tissue, travel the central lumen, and exit via the blood signal port disposed externally of the tissue.

2. The surgical assembly of claim 1, wherein the deployable coupler defines a central coupler lumen in fluid communication with the central lumen.

3. The surgical assembly of claim 2, wherein the introducer sheath, the actuator, and the deployable coupler are deliverable into a body lumen via a guidewire configured to extend through the central lumen of the introducer sheath, the elongate shaft of the actuator, and the central coupler lumen of the deployable coupler.

4. The surgical assembly of claim 2, further comprising a plug tool 70 configured to deploy a plug tool within the central coupler lumen of the deployable coupler to prevent fluid flow therethrough.

5. The surgical assembly of claim 1, wherein the deployable coupler further comprises a press ring centrally disposed between the proximal slits and the distal slits.

6. The surgical assembly of claim 1, wherein actuator is configured to deploy the proximal wing via a torsion force applied in a first rotational direction to the deployable coupler.

7. The surgical assembly of claim 6, wherein the actuator is configured to deploy the distal wing via a torsion force applied in a second rotational direction to the deployable coupler opposite the first direction.

8. The surgical assembly of claim 1, wherein the blood signal inlet is disposed proximally of the proximal slits.

9. The surgical assembly of claim 1, wherein the proximal wing and the distal wing are reversibly deployable.

10. A surgical assembly, comprising:
an actuator having an elongate shaft and an outer shaft disposed around the elongate shaft, the outer shaft being slidable relative to the elongate shaft, the outer shaft abutting a slide plate disposed at a distal end of the outer shaft; and
a deployable coupler coupled to a distal end of the elongate shaft and at a position distal of the slide plate;
wherein the outer shaft is configured to slide distally to cause the slide plate to join with the deployable coupler and capture tissue therebetween; and
and wherein the actuator is configured to eject the joined deployable coupler and the slide plate as a unit.

11. The surgical assembly of claim 10, wherein the deployable coupler comprises a plurality of distal slits formed therein configured to form a distal wing.

12. The surgical assembly of claim 10, wherein the outer shaft comprises a proximal region and a distal region, and wherein a diameter of the distal region is greater than a diameter of the proximal region.

13. The surgical assembly of claim 12, wherein the diameter of the distal region is substantially equal to a diameter of the slide plate.

14. The surgical assembly of claim 12, wherein the distal region flares outward from the proximal region.

15. The surgical assembly of claim 10, wherein the deployable coupler defines a central coupler lumen through which fluid is configured to flow.

16. The surgical assembly of claim 10, wherein the elongate shaft defines a central lumen therethrough.

17. The surgical assembly of claim 16, wherein the deployable coupler defines a central coupler lumen, and wherein the central lumen and the central coupler lumen are configured to receive a penetrator therethrough.

18. The surgical assembly of claim 17, wherein the penetrator is configured to extend beyond a distal end of the deployable coupler and to penetrate tissue and create an opening therein such that the deployable coupler can be advanced through the opening.

19. The surgical assembly of claim 10, wherein actuator is configured to deploy the distal wing via torsion and compressive forces applied in a first rotational direction to the deployable coupler.

20. The surgical assembly of claim 19, wherein the actuator is configured to deploy the slide plate via a torsion force applied in a second rotational direction to the deployable coupler opposite the first direction and/or a longitudinal force applied to the deployable coupler.
